SEQUENCE LISTING (99 sequences)

SEQ ID NO: 1 - DNA, 13 bp, Artificial Sequence
  synthetic partially double-stranded oligonucleotide first strand
nrngttgtaacna

SEQ ID NO: 2 - DNA, 14 bp, Artificial Sequence
  synthetic partially double-stranded oligonucleotide first strand
tngttacaacntnc

SEQ ID NO: 3 - DNA, 8 bp, Artificial Sequence
  synthetic first strand complementary sequence
gttgtaac

SEQ ID NO: 4 - DNA, 8 bp, Artificial Sequence
  synthetic second strand complementary sequence
gttacaac

SEQ ID NO: 5 - protein, 309 aa, Escherichia coli
  Ter binding polypeptide
MARYDLVDRLNTTFRQMEQELAAFAAHLEQHKLLVARVFSLPEVKKEDEHNPLNRIEVKQHLGNDAQSQALRHFRHLFIQQQSENRSSKAAVRLPGVLCYQVDNLSQAALVSHIQHINKLKTTFEHIVTVESELPTAARFEWVHRHLPGLITLNAYRTLTVLHDPATLRFGWANKHIIKNLHRDEVLAQLEKSLKSPRSVAPWTREEWQRKLEREYQDIAALPQNAKLKIKRPVKVQPIARVWYKGDQKQVQHACPTPLIALINRDNGAGVPDVGELLNYDADNVQHRYKPQAQPLRLIIPRLHLYVAD

SEQ ID NO: 6 - DNA, 23 bp, Artificial Sequence
  synthetic TerB oligonucleotide top strand sequence
aataagtatgttgtaactaaagt

SEQ ID NO: 7 - DNA, 23 bp, Artificial Sequence
  synthetic TerA oligonucleotide top strand sequence
aattagtatgttgtaactaaagt

SEQ ID NO: 8 - DNA, 23 bp, Artificial Sequence
  synthetic TerC oligonucleotide top strand sequence
atataggatgttgtaactaatat

SEQ ID NO: 9 - DNA, 23 bp, Artificial Sequence
  synthetic TerD oligonucleotide top strand sequence
cattagtatgttgtaactaaatg

SEQ ID NO: 10 - DNA, 23 bp, Artificial Sequence
  synthetic TerE oligonucleotide top strand sequence
ttatagtatgttgtaactaagca

SEQ ID NO: 11 - DNA, 23 bp, Artificial Sequence
  synthetic TerF oligonucleotide top strand sequence
ccttcgtatgttgtaacgacgat

SEQ ID NO: 12 - DNA, 23 bp, Artificial Sequence
  synthetic TerG oligonucleotide top strand sequence
gtcaaggatgttgtaactaacca

SEQ ID NO: 13 - DNA, 23 bp, Artificial Sequence
  synthetic TerH oligonucleotide top strand sequence
cgatcgtatgttgtaactatctc

SEQ ID NO: 14 - DNA, 23 bp, Artificial Sequence
  synthetic TerI oligonucleotide top strand sequence
aacatggaagttgtaactaaccg

SEQ ID NO: 15 - DNA, 23 bp, Artificial Sequence
  synthetic TerJ oligonucleotide top strand sequence
acgcagaaagttgtaactaatgc

SEQ ID NO: 16 - DNA, 21 bp, Artificial Sequence
  synthetic biotyinylated TerB oligonucleotide first strand sequence
ataagtatgttgtaactaaag

SEQ ID NO: 17 - DNA, 21 bp, Artificial Sequence
  synthetic TerB oligonucleotide second strand sequence
ctttagttacaacatacttat

SEQ ID NO: 18 - DNA, 21 bp, Artificial Sequence
  synthetic rTerB oligonucleotide first strand sequence
ataagtatgttgtaactaaag

SEQ ID NO: 19 - DNA, 21 bp, Artificial Sequence
  synthetic biotinylated rTerB oligonucleotide second strand sequence
ctttagttacaacatacttat

SEQ ID NO: 20 - DNA, 21 bp, Artificial Sequence
  synthetic F2p-rTerB oligonucleotide first strand sequence
ataagtatgttgtaactaacc

SEQ ID NO: 21 - DNA, 21 bp, Artificial Sequence
  synthetic F3p-rTerB oligonucleotide first strand sequence
ataagtatgttgtaactaccc

SEQ ID NO: 22 - DNA, 21 bp, Artificial Sequence
  synthetic F3p-TerB oligonucleotide second strand sequence
gggtagttacaacatacttat

SEQ ID NO: 23 - DNA, 21 bp, Artificial Sequence
  synthetic F4p-rTerB oligonucleotide first strand sequence
ataagtatgttgtaactcccc

SEQ ID NO: 24 - DNA, 21 bp, Artificial Sequence
  synthetic F4p-TerB oligonucleotide second strand sequence
ggggagttacaacatacttat

SEQ ID NO: 25 - DNA, 21 bp, Artificial Sequence
  synthetic F3n-TerB oligonucleotide second strand sequence
ctttagttacaacatactccc

SEQ ID NO: 26 - DNA, 21 bp, Artificial Sequence
  synthetic F3n-rTerB oligonucleotide first strand sequence
gggagtatgttgtaactaaag

SEQ ID NO: 27 - DNA, 21 bp, Artificial Sequence
  synthetic F4n-TerB oligonucleotide second strand sequence
ctttagttacaacataccccc

SEQ ID NO: 28 - DNA, 21 bp, Artificial Sequence
  synthetic F4n-rTerB oligonucleotide first strand sequence
gggggtatgttgtaactaaag

SEQ ID NO: 29 - DNA, 21 bp, Artificial Sequence
  synthetic F5n-TerB oligonucleotide second strand sequence
ctttagttacaacatagcccc

SEQ ID NO: 30 - DNA, 21 bp, Artificial Sequence
  synthetic F5n-rTerB oligonucleotide first strand sequence
ggggctatgttgtaactaaag

SEQ ID NO: 31 - DNA, 16 bp, Artificial Sequence
  synthetic delta5-rTerB oligonucleotide first strand sequence
tatgttgtaactaaag

SEQ ID NO: 32 - DNA, 21 bp, Artificial Sequence
  synthetic F6n-rTerB oligonucleotide first strand sequence
ggggcgatgttgtaactaaag

SEQ ID NO: 33 - DNA, 21 bp, Artificial Sequence
  synthetic F7n-rTerB oligonucleotide first strand sequence
ggggcggtgttgtaactaaag

SEQ ID NO: 34 - DNA, 21 bp, Artificial Sequence
  synthetic biotinylated F5-TerB oligonucleotide first strand sequence
ggggctatgttgtaactaaag

SEQ ID NO: 35 - DNA, 21 bp, Artificial Sequence
  synthetic F5-TerB(G2) oligonucleotide second strand sequence
ctttagttacaacatacttag

SEQ ID NO: 36 - DNA, 21 bp, Artificial Sequence
  synthetic F5-TerB(G3) oligonucleotide second strand sequence
ctttagttacaacatactttt

SEQ ID NO: 37 - DNA, 21 bp, Artificial Sequence
  synthetic F5-TerB(G4) oligonucleotide second strand sequence
ctttagttacaacatactgat

SEQ ID NO: 38 - DNA, 21 bp, Artificial Sequence
  synthetic F5-TerB(G5) oligonucleotide second strand sequence
ctttagttacaacatacgtat

SEQ ID NO: 39 - DNA, 21 bp, Artificial Sequence
  synthetic F5-TerB(C6) oligonucleotide second strand sequence
ctttagttacaacataattat

SEQ ID NO: 40 - DNA, 17 bp, Artificial Sequence
  synthetic delta4p-rTerB oligonucleotide first strand sequence
ataagtatgttgtaact

SEQ ID NO: 41 - DNA, 17 bp, Artificial Sequence
  synthetic delta4p-TerB oligonucleotide second strand sequence
agttacaacatacttat

SEQ ID NO: 42 - DNA, 18 bp, Artificial Sequence
  synthetic delta3n-TerB oligonucleotide second strand sequence
ctttagttacaacatact

SEQ ID NO: 43 - DNA, 18 bp, Artificial Sequence
  synthetic delta3n-rTerB oligonucleotide first strand sequence
agtatgttgtaactaaag

SEQ ID NO: 44 - DNA, 17 bp, Artificial Sequence
  synthetic single O/H C oligonucleotide second strand sequence
ctttagttacaacatac

SEQ ID NO: 45 - DNA, 21 bp, Artificial Sequence
  synthetic Bromo-TerB oligonucleotide second strand sequence
cttnagttacaacanacttat

SEQ ID NO: 46 - DNA, 21 bp, Artificial Sequence
  synthetic Iodo-TerB oligonucleotide second strand sequence
cttnagttacaacanacttat

SEQ ID NO: 47 - DNA, 35 bp, Artificial Sequence
  synthetic Ext-rTerB oligonucleotide first strand sequence
gcagccagctccgaataagtatgttgtaactaaag

SEQ ID NO: 48 - DNA, 35 bp, Artificial Sequence
  synthetic biotinylated Ext-rTerB oligonucleotide second strand sequence
ctttagttacaacatacttattcggagctggctgc

SEQ ID NO: 49 - DNA, 35 bp, Artificial Sequence
  synthetic 1 mismatch oligonucleotide first strand sequence
gcagccagctccgaataattatgttgtaactaaag

SEQ ID NO: 50 - DNA, 35 bp, Artificial Sequence
  synthetic 2 mismatch oligonucleotide first strand sequence
gcagccagctccgaatacttatgttgtaactaaag

SEQ ID NO: 51 - DNA, 35 bp, Artificial Sequence
  synthetic 3 mismatch oligonucleotide first strand sequence
gcagccagctccgaatccttatgttgtaactaaag

SEQ ID NO: 52 - DNA, 35 bp, Artificial Sequence
  synthetic 4 mismatch oligonucleotide first strand sequence
gcagccagctccgaaaccttatgttgtaactaaag

SEQ ID NO: 53 - DNA, 35 bp, Artificial Sequence
  synthetic 5 mismatch oligonucleotide first strand sequence
gcagccagctccgaaacctcatgttgtaactaaag

SEQ ID NO: 54 - DNA, 34 bp, Artificial Sequence
  synthetic flipped C6 oligonucleotide first strand sequence
gcagccagctccgaataatatgttgtaactaaag

SEQ ID NO: 55 - DNA, 19 bp, Artificial Sequence
  synthetic oligonucleotide first strand
nnnnnnrngttgtaacnan

SEQ ID NO: 56 - DNA, 19 bp, Artificial Sequence
  synthetic oligonucleotide second strand
ntngttacaacntncnnnn

SEQ ID NO: 57 - DNA, 35 bp, Artificial Sequence
  synthetic oligonucleotide first strand
nnnnnnnnnnnnnnnnnnnnrngttgtaacnannn

SEQ ID NO: 58 - DNA, 35 bp, Artificial Sequence
  synthetic oligonucleotide first strand
nnnnnnnnnnnnnnnnnnnnrtgttgtaactaaag

SEQ ID NO: 59 - DNA, 35 bp, Artificial Sequence
  synthetic oligonucleotide second strand
nnntagttacaacatacnnnnnnnnnnnnnnnnnn

SEQ ID NO: 60 - DNA, 35 bp, Artificial Sequence
  synthetic oligonucleotide second strand
ctttagttacaacatacnnnnnnnnnnnnnnnnnn

SEQ ID NO: 61 - DNA, 15 bp, Artificial Sequence
  synthetic oligonucleotide
nnangttgtaacnan

SEQ ID NO: 62 - DNA, 16 bp, Artificial Sequence
  synthetic oligonucleotide
nnnangttgtaacnan

SEQ ID NO: 63 - DNA, 17 bp, Artificial Sequence
  synthetic oligonucleotide
nnnnangttgtaacnan

SEQ ID NO: 64 - DNA, 16 bp, Artificial Sequence
  synthetic oligonucleotide
nnnangttgtaacnan

SEQ ID NO: 65 - DNA, 17 bp, Artificial Sequence
  synthetic oligonucleotide
nnnnangttgtaacnan

SEQ ID NO: 66 - DNA, 18 bp, Artificial Sequence
  synthetic oligonucleotide
nnnnnangttgtaacnan

SEQ ID NO: 67 - DNA, 17 bp, Artificial Sequence
  synthetic oligonucleotide
nnnnangttgtaacnan

SEQ ID NO: 68 - DNA, 18 bp, Artificial Sequence
  synthetic oligonucleotide
nnnnnangttgtaacnan

SEQ ID NO: 69 - DNA, 16 bp, Artificial Sequence
  synthetic oligonucleotide
ntngttacaacntncn

SEQ ID NO: 70 - DNA, 17 bp, Artificial Sequence
  synthetic oligonucleotide
ntngttacaacntncnn

SEQ ID NO: 71 - DNA, 18 bp, Artificial Sequence
  synthetic oligonucleotide
ntngttacaacntncnnn

SEQ ID NO: 72 - DNA, 17 bp, Artificial Sequence
  synthetic oligonucleotide
ntngttacaacntncnn

SEQ ID NO: 73 - DNA, 18 bp, Artificial Sequence
  synthetic oligonucleotide
ntngttacaacntncnnn

SEQ ID NO: 74 - DNA, 19 bp, Artificial Sequence
  synthetic oligonucleotide
ntngttacaacntncnnnn

SEQ ID NO: 75 - DNA, 60 bp, Artificial Sequence
  synthetic oligonucleotide
taatggctggtctgaacgacatcttcgaagctcagaaaatcgaatggcacgaacatatga

SEQ ID NO: 76 - DNA, 62 bp, Artificial Sequence
  synthetic oligonucleotide
cgcgtcatatgttcgtgccattcgattttctgagcttcgaagatgtcgttcagaccagccat

SEQ ID NO: 77 - DNA, 21 bp, Artificial Sequence
  synthetic oligonucleotide
ggggctatgttgtaactaaag

SEQ ID NO: 78 - DNA, 21 bp, Artificial Sequence
  synthetic oligonucleotide
tattcatacaacattgatttc

SEQ ID NO: 79 - DNA, 71 bp, Artificial Sequence
  synthetic oligonucleotide
ggggctatgttgtaactaaagtttttttttttttttttttttttttttttttttttttttttttttttttt

SEQ ID NO: 80 - DNA, 71 bp, Artificial Sequence
  synthetic oligonucleotide
tttttttttttttttttttttttttttttttttttttttttttttttttttattcatacaacattgatttc

SEQ ID NO: 81 - DNA, 16 bp, Artificial Sequence
  synthetic oligonucleotide
ttagttacaacatact

SEQ ID NO: 82 - DNA, 16 bp, Artificial Sequence
  synthetic oligonucleotide
tgatatgttgtaacta

SEQ ID NO: 83 - DNA, 37 bp, Artificial Sequence
  synthetic oligonucleotide
aataagtatgttgtaactaaagtggatcaattcataa

SEQ ID NO: 84 - DNA, 37 bp, Artificial Sequence
  synthetic oligonucleotide
ttattcatacaacattgatttcacctagttaagtatt

SEQ ID NO: 85 - DNA, 37 bp, Artificial Sequence
  synthetic oligonucleotide
gggggctatgttgtaactaaagtggatcaattcataa

SEQ ID NO: 86 - DNA, 37 bp, Artificial Sequence; identical to SEQ ID NO: 84
  synthetic oligonucleotide

SEQ ID NO: 87 - DNA, 37 bp, Artificial Sequence
  synthetic oligonucleotide
tatgttgtaactaaagtggatcaattcataaaataag

SEQ ID NO: 88 - DNA, 38 bp, Artificial Sequence
  synthetic oligonucleotide
catacaacattgatttcacctagttaagtattttattc

SEQ ID NO: 89 - protein, 11 aa, Artificial Sequence
  synthetic human c-Myc 9E10 epitope
EQKLISEEDLN

SEQ ID NO: 90 - protein, 6 aa, Artificial Sequence
  synthetic C-terminal His6 tag, 6-HIS
HHHHHH

SEQ ID NO: 91 - protein, 5 aa, Artificial Sequence
  synthetic sortase recognition sequence
LPETG

SEQ ID NO: 92 - protein, 4 aa, Artificial Sequence
  synthetic flexible linker, repeated an undefined number of times
GGGS

SEQ ID NO: 93 - DNA, 21 bp, Artificial Sequence
  synthetic RSC838 oligonucleotide
ggggctatgttgtaactaaag

SEQ ID NO: 94 - DNA, 21 bp, Artificial Sequence
  synthetic RSC1044 oligonucleotide
ctttagttacaacatacttat

SEQ ID NO: 95 - DNA, 21 bp, Artificial Sequence
  synthetic RSC1246 oligonucleotide
ggggaaatgttgtaactaaag

SEQ ID NO: 96 - DNA, 21 bp, Artificial Sequence
  synthetic RSC1249 oligonucleotide
ctttagttacaacatncttat

SEQ ID NO: 97 - DNA, 19 bp, Artificial Sequence
  synthetic PSJCU1 oligonucleotide
cagtatggtgcttcacacg

SEQ ID NO: 98 - DNA, 58 bp, Artificial Sequence
  synthetic PSJCU2 oligonucleotide
cagtatggtgcttcacacggatagatgttacttcgctctttagttacaacatacttat

SEQ ID NO: 99 - DNA, 20 bp, Artificial Sequence
  synthetic PSJCU3 oligonucleotide
tatgttgtaactaaagagcg

(19) United States
(12) Patent Application Publication
(10) Pub. No.: US 2011/0189664 A1
(43) Pub. Date: Aug. 4, 2011

(54) DIAGNOSTICS IN A MONOPLEX/MULTIPLEX FORMAT

(75) Inventors: Nicholas Edward Dixon, Austinmer (AU); Patrick Marcel Schaeffer, Alice River (AU)

(73) Assignee: University of Wollongong

(21) Appl. No.: 12/602,995

(22) PCT Filed: Jun. 6, 2007
(86) PCT No.: PCT/AU07/00798

§ 371 Date: Jul. 23, 2010

(51) Int. Cl. C12Q 1/68

(52) U.S. Cl. 435/6.11

(57) ABSTRACT

The present invention relates to a method of detecting and/or quantifying a target molecule from a sample obtained from a subject wherein the method comprises: (i) incubating a fusion protein or conjugate comprising a Ter binding polypeptide fused to at least one anti-target molecule or fragment thereof with a partially double-stranded oligonucleotide for a time and under conditions sufficient to bind to said Ter binding polypeptide thereby producing a complex; (ii) incubating said complex in the presence of said sample comprising said target molecule for a time and under conditions sufficient for said anti-target molecule to bind to said target molecule thereby producing a target-bound complex; (iii) incubating said target-bound complex in the presence of at least one immobilised molecule wherein said immobilised molecule has an affinity to said target molecule; (iv) incubating said immobilised molecule for a time and under conditions sufficient to bind to said target molecule thus immobilising said target molecule; and (v) detecting and/or quantifying said target molecule.

### FIELD OF THE INVENTION

The present invention relates to the use of a Tus-Ter derivative complex as a linking system between an anti target protein and a DNA fragment for the early detection of a disease or condition. The present invention further relates to the application of the Tus-Ter derivative complex as a diagnostic tool for the early detection of a disease or condition.

### BACKGROUND

In most cases, the earlier a disease is detected the better the outcome for the patient. This is particularly true for cancer, and also for infectious diseases, to avoid epidemic outbreaks or pandemics. Another important drive in diagnostics research is to improve current techniques to achieve better reproducibility and quantitative detection, for example, of hormones and drugs (Stenman, Clin Chem 51:801-802, 2005). Most molecular screening diagnostics are based on immunoassay methods where a biomarker is detected and quantified. The most common methods are ELISA (Engvall & Perlman, Immunochemistry 8:871-874, 1971) or derivatives of ELISA, which are not very sensitive. Whatever the format of the assay, these detection systems are ultimately based on an antibody (Ab) physically linked to some sort of device that detects an antigen (Ag).

In the last decade there has been a push to develop new highly-sensitive methods such as ultrasensitive immunoassay techniques which are leading to the discovery of new biomarkers that are highly specific and appear very early in the development of, for example, a particular cancer or infectious disease. Among these, ImmunoPCR, which detects the antibody by PCR amplification of a conjugated DNA molecule, is very promising. However, it is in general very cumbersome to perform (Niemeyer et al., Trends Biotechnol 23:208-216, 2005; Barletta, Mol Aspects Med 27:224-253, 2006). A common problem in immunoassays is that all of the different molecular interactions used in the assay need to be stable to avoid poor detection levels. Bleeding of the reagents or biomarker during successive washing steps as well as non-specific interactions caused by the detector molecule need to be reduced, since they can lead to poor detection limits or false positives. In consequence, the number of sub-optimal interactions in the system needs to be minimized to achieve the best possible signal detection. One way to reduce loss of signal is to ensure that all non-covalent interactions (e.g., antibody-antigen) used in the system are very strong and exhibit the slowest possible off-rates, and that the signal generation system is firmly attached to the Ab domain (Dhawan, Expert Rev Mol Diagn 6:749-760, 2006).

The invention described herein refers to a new technology platform for development of diagnostics capable of detecting disease markers at very low concentrations. Specifically, the inventors have discovered that the use of TT-Lock DNA (incorporated herein by reference to WO 2006/081623 in its entirety) in a monoplex or multiplex system allows for a sensitive and specific diagnostic tool for detecting various targets such as disease biomarkers.

### SUMMARY

According to a first aspect of the present invention, there is provided a method of detecting and/or quantifying a target molecule from a sample obtained from a subject wherein the method comprises:
(i) incubating a fusion protein or conjugate comprising a Ter binding polypeptide fused to at least one anti-target molecule or fragment thereof with a partially double-stranded oligonucleotide for a time and under is conditions sufficient to bind to said Ter binding polypeptide thereby producing a complex;
(ii) incubating said complex in the presence of said sample comprising said target molecule for a time and under conditions sufficient for said anti-target molecule to bind to said target molecule thereby producing a target-bound complex;
(iii) incubating said target-bound complex in the presence of at least one immobilised molecule wherein said immobilised molecule has an affinity to said target molecule;
(iv) incubating said immobilised molecule for a time and under conditions sufficient to bind to said target molecule thus immobilising said target molecule; and
(v) detecting and/or quantifying said target molecule.

According to a second aspect of the present invention, there is provided a method of detecting and/or quantifying a target molecule from a sample obtained from a subject wherein the method comprises:
(i) incubating a fusion protein or conjugate comprising a Ter binding polypeptide fused to at least one anti-target molecule or fragment thereof in the presence of said sample comprising said target molecule for a time and under conditions sufficient for said anti-target molecule to bind to said target molecule thereby producing a target-bound complex;
(ii) incubating said target-bound complex with a partially double-stranded oligonucleotide for a time and under conditions sufficient to bind to said Ter binding polypeptide thereby producing a target/oligonucleotide-bound complex;
(iii) incubating said target/oligonucleotide-bound complex in the presence of at least one immobilised molecule wherein said immobilised molecule has an affinity to said target molecule and for a time and under conditions io sufficient to bind to said target molecule thus immobilising said target molecule; and
(iv) detecting and/or quantifying said target molecule.

According to a third aspect of the present invention, there is provided a method of detecting and/or quantifying a target molecule from a sample obtained from a subject wherein the method comprises:
(i) incubating a fusion protein or conjugate comprising a Ter binding polypeptide fused to at least one anti-target molecule or fragment thereof in the presence of said sample comprising said target molecule for a time and under conditions sufficient for said anti-target molecule to bind to said target molecule thereby producing a target-bound complex;
(ii) incubating said target-bound complex in the presence of at least one immobilised molecule wherein said immobilised molecule has an affinity to said target molecule for a time and under conditions sufficient to bind to said immobilised molecule thereby producing an immobilised target-bound complex;
(iii) incubating said immobilised target-bound complex with a partially double- stranded oligonucleotide for a time and under conditions sufficient to bind to said Ter binding polypeptide;
(iv) detecting and/or quantifying said target molecule.

According to a fourth aspect of the present invention, there is provided a method of detecting and/or quantifying a target molecule from a sample obtained from a subject wherein the method comprises:
(i) incubating a fusion protein or conjugate comprising a Ter binding polypeptide fused to a first target molecule or fragment thereof with a partially double-stranded oligonucleotide for a time and under conditions sufficient to bind to said Ter binding polypeptide thereby producing a complex;
(ii) incubating said complex in the presence of said sample comprising a second target molecule for a time and under conditions sufficient for said complex and said second target molecule to compete for binding to an anti-target molecule thereby producing an anti-target-molecule-bound complex;
(iii) incubating anti-target-molecule-bound complex in the presence of at least one immobilised molecule wherein said immobilised molecule has an affinity to said anti-target-molecule;
(iv) incubating said immobilised molecule for a time and under conditions sufficient to bind to said anti-target-molecule thus immobilising said anti-target-molecule; and
(v) detecting and/or quantifying at least one target molecule.

According to a fifth aspect of the present invention, there is provided a method of screening a sample obtained from a subject for the presence of at least one target molecule wherein the method comprises:
(i) incubating a fusion protein or conjugate comprising a Ter binding polypeptide fused to at least one anti-target molecule or fragment thereof with a partially double-stranded oligonucleotide for a time and under conditions sufficient to bind to said Ter binding polypeptide thereby producing a complex;
(ii) incubating said complex in the presence of said sample comprising said target molecule for a time and under conditions sufficient for said anti-target molecule to bind to said target molecule thereby producing a target-bound complex;
(iii) incubating said target-bound complex in the presence of at least one immobilised molecule wherein said immobilised molecule has an affinity to said target molecule;
(iv) incubating said immobilised molecule for a time and under conditions sufficient to bind to said target molecule thus immobilising said target molecule; and
(v) detecting and/or quantifying at least one target molecule.

According to a sixth aspect of the present invention, there is provided a method of screening a sample obtained from a subject for the presence of at least one target molecule wherein the method comprises:
(i) incubating a fusion protein or conjugate comprising a Ter binding polypeptide fused to at least one anti-target molecule or fragment thereof in the presence of said sample comprising said target molecule for a time and under conditions sufficient for said anti-target molecule to bind to said target molecule thereby producing a target-bound complex;
(ii) incubating said target-bound complex with a partially double-stranded oligonucleotide for a time and under conditions sufficient to bind to said Ter binding polypeptide thereby producing a target/oligonucleotide-bound complex;
(iii) incubating said target/oligonucleotide-bound complex in the presence of at least one immobilised molecule wherein said immobilised molecule has is an affinity to said target molecule and for a time and under conditions sufficient to bind to said target molecule thus immobilising said target molecule; and
(iv) detecting and/or quantifying at least one target molecule.

According to a seventh aspect of the present invention, there is provided a method of screening a sample obtained from a subject for the presence of at least one target molecule wherein the method comprises:
(i) incubating a fusion protein or conjugate comprising a Ter binding polypeptide fused to at least one anti-target molecule or fragment thereof in the presence of said sample comprising said target molecule for a time and under conditions sufficient for said anti-target molecule to bind to said target molecule thereby producing a target-bound complex;
(ii) incubating said target-bound complex in the presence of at least one immobilised molecule wherein said immobilised molecule has an affinity to said target molecule for a time and under conditions sufficient to bind to said immobilised molecule thereby producing an immobilised target- bound complex;
(iii) incubating said immobilised target-bound complex with a partially double-stranded oligonucleotide for a time and under conditions sufficient to bind to said Ter binding polypeptide;
(iv) detecting and/or quantifying at least one target molecule.

According to an eighth aspect of the present invention, there is provided a method of screening a sample obtained from a subject for the presence of at least one target molecule wherein the method comprises:
(i) incubating a fusion protein or conjugate comprising a Ter binding polypeptide fused to a first target molecule or fragment thereof with a partially double-stranded oligonucleotide for a time and under conditions sufficient to bind to said Ter binding polypeptide thereby producing a complex;
(ii) incubating said complex in the presence of said sample comprising a second target molecule for a time and under conditions sufficient for said complex and said second target molecule to compete for binding to an anti-target molecule thereby producing an anti-target-molecule-bound complex;
(iii) incubating anti-target-molecule-bound complex in the presence of at least one immobilised molecule wherein said immobilised molecule has an affinity to said anti-target-molecule;
(iv) incubating said immobilised molecule for a time and under conditions sufficient to bind to said anti-target-molecule thus immobilising said anti-target-molecule; and
(v) detecting and/or quantifying at least one target molecule.

The method may comprise the use of an ELISA and/or a PCR. The ELISA may be a direct, indirect or sandwich ELISA. The ELISA may be a competitive or non-competitive ELISA.

According to a ninth aspect of the present invention, there is provided a process of identifying at least one target molecule from a sample obtained from a subject, wherein said process comprises:
(i) incubating a fusion protein or conjugate comprising a Ter binding polypeptide fused to at least one anti-target molecule or fragment thereof with a partially double-stranded oligonucleotide for a time and under conditions sufficient to bind to said Ter binding polypeptide thereby producing a complex;
(ii) incubating said complex in the presence of said sample comprising said target molecule for a time and under conditions sufficient for said anti-target molecule to bind to said target molecule thereby producing a target-bound complex;
(iii) incubating said target-bound complex in the presence of at least one immobilised molecule wherein said immobilised molecule has an affinity to said target molecule;
(iv) incubating said immobilised molecule for a time and under conditions sufficient to bind to said target molecule thus immobilising said target molecule; and
(v) detecting and/or quantifying said target molecule.

According to a tenth aspect of the present invention, there is provided a process of identifying at least one target molecule from a sample obtained from a subject, wherein said process comprises:
(i) incubating a fusion protein or conjugate comprising a Ter binding polypeptide fused to at least one anti-target molecule or fragment thereof in the presence of said sample comprising said target molecule for a time and under conditions sufficient for said anti-target molecule to bind to said target molecule thereby producing a target-bound complex;
(ii) incubating said target-bound complex with a partially double-stranded oligonucleotide for a time and under conditions sufficient to bind to said Ter binding polypeptide thereby producing a target/oligonucleotide-bound complex;
(iii) incubating said target/oligonucleotide-bound complex in the presence of at least one immobilised molecule wherein said immobilised molecule has an affinity to said target molecule and for a time and under conditions sufficient to bind to said target molecule thus immobilising said target molecule; and
(iv) detecting and/or quantifying said target molecule. According to a eleventh aspect of the present invention, there is provided a process of identifying at least one target molecule from a sample obtained from a subject, wherein said process comprises:
(I) incubating a fusion protein or conjugate comprising a Ter binding polypeptide fused to at least one anti-target molecule or fragment thereof in the presence of said sample comprising said target molecule for a time and under conditions sufficient for said anti-target molecule to bind to said target molecule thereby producing a target-bound complex;
(ii) incubating said target-bound complex in the presence of at least one immobilised molecule wherein said immobilised molecule has an affinity to said target molecule for a time and under conditions sufficient to bind to said immobilised molecule thereby producing an immobilised target- bound complex;
(iii) incubating said immobilised target-bound complex with a partially double-stranded oligonucleotide for a time and under conditions sufficient to bind to said Ter binding polypeptide;
(iv) detecting and/or quantifying said target molecule.

According to a twelfth aspect of the present invention, there is provided a process of identifying at least one target molecule from a sample obtained from a subject, wherein said process comprises:
(i) incubating a fusion protein or conjugate comprising a Ter binding polypeptide fused to a first target molecule or fragment thereof with a partially double-stranded oligonucleotide for a time and under conditions sufficient to bind to said Ter binding polypeptide thereby producing a complex;
(ii) incubating said complex in the presence of said sample comprising a second target molecule for a time and under conditions sufficient for said complex and said second target molecule to compete for binding to an anti-target molecule thereby producing an anti-target-molecule-bound complex;
(iii) incubating anti-target-molecule-bound complex in the presence of at least one immobilised molecule wherein said immobilised molecule has an affinity to said anti-target-molecule;
(iv) incubating said immobilised molecule for a time and under conditions sufficient to bind to said anti-target-molecule thus immobilising said anti-target-molecule; and
(v) detecting and/or quantifying at least one target molecule.

The process may comprise the use of an ELISA and/or a PCR. The ELISA may be a direct, indirect or sandwich ELISA. The ELISA may be a competitive or non-competitive ELISA.

It is submitted herein that a skilled addressee would not limit the processes and methods of the invention to the order in which the steps identified as (ii) to (iv) are listed in the above aspects.

According to a thirteenth aspect of the present invention, there is provided a kit for detecting a target molecule from a sample of a subject in a monoplex or multiplex format comprising a first strand oligonucleotide or an analogue or derivative thereof, and a second strand oligonucleotide or an analogue or derivative thereof, wherein said first strand oligonucleotide or analogue or derivative and said second strand oligonucleotide or analogue or derivative are in a form suitable for their annealing to produce a partially double-stranded oligonucleotide wherein:

(a) said first strand comprises the sequence:

| | |
|---|---|
| 5′-N_{C} R N_{D} G T T G T A A C N_{D} A-3′ | (SEQ ID NO: 1) |

or an analogue or derivative of said sequence; and

(b) said second strand comprises the sequence:

| | |
|---|---|
| 5′-T N_{D} G T T A C A A C N_{D} T N_{C} C-3′ | (SEQ ID NO: 2) |

or an analogue or derivative of said sequence
   wherein R is a purine, N_{C} and N_{D} are each a DNA or RNA residue or analogue thereof, N_{D} residues in said first strand and said second strand are sufficiently complementary to permit said ND residues to be annealed in the double-stranded oligonucleotide, and the sequence 5′-GTTGTAAC-3′ (SEQ ID NO: 3) of said first strand is annealed to the complementary sequence 5′-GTTACAAC-3′ (SEQ ID NO: 4) of said second strand.

According to a fourteenth aspect of the present invention, there is provided a kit for detecting a target molecule from a sample obtained from a subject in a monoplex or multiplex format, wherein said kit comprises a first strand oligonucleotide or an analogue or derivative thereof, and a second strand oligonucleotide or an analogue or derivative thereof, wherein said first strand oligonucleotide or analogue or derivative and said second strand oligonucleotide or analogue or derivative are in a form suitable for their annealing to produce a partially double-stranded oligonucleotide wherein:

(a) said first strand comprises the sequence:

| | |
|---|---|
| 5′-N_{C} R N_{D} G T T G T A A C N_{D} A-3′ | (SEQ ID NO: 1) |

or an analogue or derivative of said sequence; and

(b) said second strand comprises the sequence:

(SEQ ID NO: 2)
5′-T N_{D} G T T A C A A C N D T N_{C} C-3′

or an analogue or derivative of said sequence

wherein R is a purine, N_{C} and N_{D} are each a DNA or RNA residue or analogue thereof, N_{D} residues in said first strand and said second strand are sufficiently complementary to permit said ND residues to be annealed in the double-stranded oligonucleotide, and the sequence 5′-GTTGTAAC-3′ (SEQ ID NO: 3) of said first strand is annealed to the complementary sequence 5′-GTTACAAC-3′ (SEQ ID NO: 4) of said second strand in a form suitable for conjugating to a second molecule, wherein said second molecule comprises a nucleic acid, polypeptide or small molecule.

The second molecule can be a Ter binding polypeptide.

According to any one of the preceding aspects, the double-stranded oligonucleotide may comprise a first strand and a second strand, wherein:

(a) said first strand comprises the sequence:

| | |
|---|---|
| 5′-N_{C} R N_{D} G T T G T A A C N_{D} A-3′ | (SEQ ID NO: 1) |

or an analogue or derivative of said sequence; and

(b) said second strand comprises the sequence:

| | |
|---|---|
| 5′-T N_{D} G T T A C A A C N_{D} T N_{C} C-3′ | (SEQ ID NO: 2) |

or an analogue or derivative of said sequence

wherein R is a purine, N_{C} and N_{D} are each a DNA or RNA residue or analogue thereof, N_{D} residues in said first strand and said second strand are sufficiently complementary to permit said N_{D} residues to be annealed in the double-stranded oligonucleotide, and the sequence 5′-2o GTTGTAAC-3′ (SEQ ID NO: 3) of said first strand is annealed to the complementary sequence 5′-GTTACAAC-3′ (SEQ ID NO: 4) of said second strand.

In one embodiment according to any one of the above aspects, the target molecule may be a biological marker (biomarker).

In a further embodiment, the biomarker may be a marker for the detection or indication of a disease or condition. The biomarker may be PSA.

The disease or condition may result from, or be otherwise associated with, infection of the subject caused by a viral or bacterial pathogen. The viral pathogen may be HIV.

The disease or condition may be a neurodegenerative disease or a cancer such that the neurodegenerative disease may be Alzheimer's or Parkinson's disease and the cancer may be prostate, ovarian, breast, lung or colon cancer.

The sample may be a biological sample. The biological sample may be blood, urine, mucous, vaginal discharge and any other secretions that may be collectable from a subject that is healthy or inflicted with a disease or condition.

In another embodiment according to any one of the above aspects, the anti-target molecule may be an antigen, antibody, or any other molecule that has an affinity to the target molecule.

The target molecule may be detected and/or quantified by use of a signal molecule bound to a Ter binding polypeptide or derivative, analogue or fragment thereof wherein the fragment possesses Ter binding activity, Ter or TTLock or derivatives or analogues thereof, and/or said anti-target molecules. The signal molecule can be a coloured compound, a fluorescent tag, an intercalating dye or a radioactive isotope or a combination thereof.

The oligonucleotide may be forked.

The oligonucleotide may further comprise at least one additional DNA or RNA residue or analogue thereof, at either or both the 5′- and 3′- ends of either or both the first and second strands.

The analogue may comprise a methylated, iodinated, brominated or biotinylated residue.

The oligonucleotide may be derivatized to include 5′- and/or 3′- insertions that do not adversely affect its ability to bind to a Ter binding polypeptide. The insertions may include the addition of mRNA and/or DNA that is to be presented or displayed.

In a further embodiment, said first strand comprises the sequence:

| | |
|---|---|
| 5′-(N_{A})ₘ N_{E} N_{E} N_{B} N_{B} N_{C} R N_{D} G T T G T A A C N_{D} A (N_{A})ₙ-3′ | (SEQ ID NO: 55) |

or an analogue or derivative of said sequence; and said second strand comprises the sequence:

| | |
|---|---|
| 5′-(N_{A})ₚ T N_{D} G T T A C A A C N_{D} T N_{C} C N_{B} N_{E} N_{E} (N_{A})_{D}-3′ | (SEQ ID NO: 56) |

or an analogue or derivative of said sequence

wherein N_{A} , N_{B} and N_{E} are each any DNA or RNA residue or analogue thereof, each of N_{A} and N_{B} is optional subject to the proviso that when any occurrence of N_{B} is present it is not base-paired to another residue, base-pairing of each of N_{C} to another residue is optional, each of N_{D} is base-paired with another residue, each of N_{E} is optional, subject to the proviso that if one or more of N_{E} is present it is not base-paired unless m=0 or o=0, m, n, o, p, are each an integer including zero, and said first strand and said second strand are of equal or unequal length.

The first strand may comprise the sequence:

| | |
|---|---|
| 5′-(N_{A})₁₋₁₅ N_{E} N_{E} N_{B} N_{B} N_{C} R N_{D} G T T G T A A C N_{D} A (N_{A})₃-3′ | (SEQ ID NO: 57) |

or an analogue or derivative of said sequence.

The first strand may comprise the sequence:

| | |
|---|---|
| 5′-(N_{A})₁₋₁₅ N_{E} N_{E} N_{B} N_{B} N_{C} R T G T T G T A A C T A A A G-3′ | (SEQ ID NO: 58) |

or an analogue or derivative of said sequence.

The second strand may comprise the sequence:

| | |
|---|---|
| 5′-(N_{A})₃ T A G T T A C A A C A T A C N_{B} N_{E} N_{E} (N_{A})₁₋₁₅-3′ | (SEQ ID NO: 59) |

or an analogue or derivative of said sequence.

The second strand may comprise the sequence:

| | |
|---|---|
| 5′-C T T T A G T T A C A A C A T A C N_{B} N_{E} N_{E}(N_{A})₁₋₁₅-3′ | (SEQ ID NO: 60) |

or an analogue or derivative of said sequence.

The oligonucleotide may bind to a Ter binding polypeptide covalently or non-covalently.

The oligonucleotide may be contained in a Barcode DNA sequence. The Ter binding polypeptide may have TerB-binding activity. The Ter binding polypeptide may comprise the sequence set forth as SEQ ID NO: 5.

The oligonucleotide may be derivatized to include 5′- and/or 3′- insertions that do not adversely affect its ability to bind to a Ter binding polypeptide. The insertions may include the addition of mRNA and/or DNA that is to be presented or displayed.

In one embodiment, the fusion protein of any one of the preceding aspects may be encoded by a polynucleotide.

In another embodiment, there is provided a vector which may comprise the polynucleotide. In yet another embodiment, the vector may be transformed in a host cell. The vector may contain a promoter. The promoter may be bacteriophage T7 or lambda promoter.

In yet another embodiment there is provided a chip, wherein said chip comprises the oligonucleotide of any one of the preceding aspects.

### Definitions

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

The term “nucleic acid molecule” as used herein refers to a single- or double- stranded polymer of deoxyribonucleotide, ribonucleotide bases or known analogues of natural nucleotides, or mixtures thereof. The term includes reference to the specified sequence as well as to the sequence complementary thereto, unless otherwise indicated. The terms “nucleic acid” and “polynucleotide” are used herein interchangeably. It will be understood that “5′ end” as used herein in relation to a nucleic acid molecule corresponds to the N-terminus of the encoded polypeptide and “3′ end” corresponds to the C-terminus of the encoded polypeptide.

The terms “nucleic acid molecule”, “polynucleotide” and “oligonucleotide” are used interchangeably herein.

In the present context, the term “anneal” or “annealed” or similar term shall be taken to mean that the first and second strands are to the extent that they have complementary sequences, base-paired to each other to form a double-stranded nucleic acid, either spontaneously under the conditions in which the double-stranded oligonucleotide is employed or other conditions known in the art to promote or permit base-pairing between complementary nucleotide residues or induced to form such base-pairing. As will be known to the skilled artisan, two complementary single polynucleotides comprising RNA and/or DNA including one or more ribonucleotide analogues and/or deoxyribonucleotide analogues will generally anneal to form a double helix or duplex. As will be known to the skilled artisan, the ability to form a duplex and/or the stability of a formed duplex depend on one or more factors including the length of a region of complementarity between the first and second strands, the percentage content of adenine and thymine in a region of complementarity between the first and second strands (i.e., “A+T content”), the incubation temperature relative to the melting temperature (Tm) of a duplex, and the salt concentration of a buffer or other solution in which the first and second strands are incubated. Generally, to promote duplex formation, the nucleic acid strands are incubated at a temperature that is at least about 1-5° C. below a Tm of a duplex that is predicted from its A+T content and length. Duplex formation can also be enhanced or stabilized by increasing the amount of a salt (e.g., NaCl, MgCl2, KCl, sodium citrate, etc), or by increasing the time period of the incubation, as described by Sambrook et al., Molecular Cloning: A Laboratory Manual , Cold Spring Harbor Laboratory Press New York, Third Edition, 2001; Hames and Higgins, Nucleic Acid Hybridization: A Practical Approach, IRL Press, Oxford,1985; Berger and Kimmel, Guide to Molecular Cloning Techniques, In: Methods in Enzymology, Vol 152, Academic Press, San Diego Calif., 1987; or Ausubel et al., Current Protocols in Molecular Biology, Wiley Interscience, ISBN 047150338,1992.

The term “deoxyribonucleotide” is an art-recognized term referring to those bases of DNA each comprising phosphate, deoxyribose and a purine or pyrimidine base selected from the group consisting of adenine (A), cytidine (C), guanine (G) and thymine (T). In the triphosphate form, deoxyribonucleotide triphosphates (dNTPs), e.g., dATP, dCTP, cGTP and TTP, are capable of being incorporated into DNA by an enzyme of DNA synthesis e.g., a DNA polymerase.

The term “ribonucleotide” is an art-recognized term referring to those bases of RNA each comprising a purine or pyrimidine base selected from the group consisting of adenine (A), cytidine (C), guanine (G) and uracil (U) linked to ribose. Ribonucleotides are capable of being incorporated into RNA by an enzyme of RNA synthesis e.g., an RNA polymerase.

As used herein in respect of nucleic acids or oligonucleotides, the term “upstream” shall be taken to mean that a stated integer e.g., a ribonucleotide, deoxyribonucleotide or analogue thereof, is positioned 5′ relative to a nucleotide sequence, albeit not necessarily at the 5′-end of said sequence or at the 5′-end of the nucleic acid containing the ribonucleotide, deoxyribonucleotide or analogue. Accordingly, a ribonucleotide, deoxyribonucleotide or analogue thereof positioned “upstream” of a nucleotide sequence may be internal by virtue of there being other residues positioned upstream of it. Alternatively, a ribonucleotide, deoxyribonucleotide or analogue thereof positioned “upstream” of a nucleotide sequence may be at the 5′-end.Similarly, the term “downstream” shall be taken to mean that a stated integer e.g., a ribonucleotide, deoxyribonucleotide or analogue thereof, is positioned 3′ relative to a nucleotide sequence, albeit not necessarily at the 3′-end of said sequence or at the 3′-end of the nucleic acid containing the ribonucleotide, deoxyribonucleotide or analogue. Accordingly, a ribonucleotide, is deoxyribonucleotide or analogue thereof positioned “downstream” of a nucleotide sequence may be internal by virtue of there being other residues positioned downstream of it. Alternatively, a ribonucleotide, deoxyribonucleotide or analogue thereof positioned “downstream” of a nucleotide sequence may be at the 3′-end.The term “5′-end” shall be taken to mean that a stated integer e.g., a ribonucleotide, deoxyribonucleotide or analogue thereof, is positioned 5′ relative to a nucleotide sequence such that it is at an end of nucleic acid containing the ribonucleotide, deoxyribonucleotide or analogue (i.e., there are no residues upstream of the stated integer).The term “3′-end” shall be taken to mean that a stated integer e.g., a ribonucleotide, deoxyribonucleotide or analogue thereof, is positioned 3′ relative to a nucleotide sequence such that it is at an end of nucleic acid containing the ribonucleotide, deoxyribonucleotide or analogue (i.e., there are no residues downstream of the stated integer).

The term “analogue” when used in relation to an oligonucleotide or residue thereof, means a compound having a physical structure that is related to a DNA or RNA molecule or residue, and preferably is capable of forming a hydrogen bond with a DNA or RNA residue or an analogue thereof (i.e., it is able to anneal with a DNA or RNA residue or an analogue thereof to form a base-pair). Such analogues may possess different chemical and biological properties to the ribonucleotide or deoxyribonucleotide residue to which they are structurally related. Analogues of the oligonucleotides of the present invention therefore include, for example, any functionally- equivalent nucleic acids that bind to a Ter binding protein and which include one or more analogues of A, C, G or T. For example, an analogue comprised of the nucleotide sequence of the first aspect may have one or more of the nucleotides A, C, G or T therein substituted for one or more nucleotide analogues. Methylated, iodinated, brominated or biotinylated residues are particularly preferred analogues. However, other analogues such as, for example, those analogues specified elsewhere herein, may also be used. Analogue as used herein with reference to a polypeptide can mean a polypeptide which is a derivative of the polypeptide of the invention, which derivative comprises addition, deletion, substitution of one or more amino acids, such that the polypeptide retains substantially the same function.

The term “derivative” when used in relation to the oligonucleotides of the present invention include any functionally-equivalent nucleic acids that bind to a Ter binding protein and to which include one or more nucleotides and/or nucleotide analogues upstream or downstream, including any fusion molecules produced integrally (e.g., by recombinant means) or added post-synthesis (e.g., by chemical means). Such fusions may comprise one or both strands of the double-stranded oligonucleotide of the invention with RNA or DNA added thereto or conjugated to a polypeptide (e.g., puromycin or other polypeptide), a small molecule (e.g., psoralen) or an antibody. Particularly preferred derivatives include mRNA or DNA conjugated to the oligonucleotide of the invention for displaying on a microwell or microarray surface or on the surface of a cell, phage, virus or in vitro.

As used herein the term “polypeptide” means a polymer made up of amino acids linked together by peptide bonds. The term “polypeptide” may be used interchangeably with the term “protein” and includes fragments, variants and analogues thereof.

The term “variant” as used herein refers to substantially similar sequences. Generally, polypeptide or polynucleotide sequence variants possess qualitative biological activity in common. Further, these polypeptide or polynucleotide sequence variants may share at least 50%, 55%, 60%, 65%, 70%, 75%, 80%, 85%, 90%, 95%, 96%, 97%, 98% or 99% sequence identity. Also included within the meaning of the term “variant” are homologues of polypeptides or polynucleotides of the invention. A homologue is typically a polypeptide or polynucleotide from a different species but sharing substantially the same biological function or activity as the corresponding polypeptide or polynucleotide disclosed herein.

The term “fragment” when used in relation to a polypeptide or polynucleotide molecule refers to a constituent of a polypeptide or polynucleotide. Typically the fragment possesses qualitative biological activity in common with the polypeptide or polynucleotide. However, fragments of a polynucleotide do not necessarily need to encode polypeptides which retain biological activity. Rather, a fragment may, for example, be useful as a hybridization probe or PCR primer. The fragment may be derived from a polynucleotide of the invention or alternatively may be synthesized by some other means, for example chemical synthesis.

The term “purified” means that the material in question has been removed from its natural environment or host, and associated impurities reduced or eliminated such that the molecule in question is the predominant species present. Thus, essentially, the term “purified” means that an object species is the predominant species present (ie., on a molar basis it is more abundant than any other individual species in the composition), and preferably a substantially purified fraction is a composition wherein the object species comprises at least about 30 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species. The terms “purified” and “isolated” may be used interchangeably.

As used herein, the term “Ter binding polypeptide” or “Ter binding protein”, which can be used interchangeably with the term “Tus”, refers to any polypeptide capable of binding to a Ter site, including a full-length naturally-occurring Ter binding polypeptide or a fragment or other derivative thereof having Ter binding activity or a variant, homologue or analogue thereof having Ter-binding activity. For example, the term “Ter binding polypeptide” and “Ths” includes any peptide, polypeptide, or protein or any homologue, analogue or derivative thereof having at least about 80% amino acid sequence identity to the amino acid sequence of E. coli Ter binding polypeptide set forth in SEQ ID NO: 5 wherein said polypeptide has Ter binding activity. Homologues of a Ter binding polypeptide may include any functionally-equivalent proteins to the Ter binding polypeptide of E. coli wherein said homologue is a naturally-occurring variant of said E.coli Tus having Ter binding activity. Tus homologues or homologues may include those Ter family proteins of fragments thereof that retain the ability to bind to a Ter site, such as those of bacteria that are capable of specifically binding to one or more DNA replication terminus sites on the host and plasmid genome and block progress of the DNA replication fork notwithstanding that it may not necessarily be capable of specifically binding to one or more DNA replication terminus sites on the host and plasmid genome and/or block progress of the DNA replication fork or function in fork arrest.

“Ter family protein” refers to a DNA replication terminus site-binding protein (Ter protein) that is capable of specifically binding to a DNA replication terminus site on the host and plasmid genome such as, for example, to block progress of a DNA replication fork. The amino acid sequences of several such homologues are known in the art, e.g., from a bacterium selected from the group consisting of: Shigella flexneri (Jin et al., Nucleic Acids Res. 30, 4432-4441, 2002); Salmonella enterica (McClelland et al., Nat. Genet. 36, 1268-1274, 2004); Salmonella typhimurium (McClelland et al., Nature 413, 852-856, 2001); Klebsiella pneumoniae (Henderson et al., Mol. Genet. Genomics 265, 941-953, 2001); Yersinia pestis (Song et al., DNA Res. 11, 179-197, 2004); and Proteus vulgaris (Murata et al., J. Bacteriol. 184, 3194-3202, 2002). Analogues of a Ter binding polypeptide may include any functionally-equivalent synthesized variants of the E. coli Ter binding polypeptide having Ter binding activity. Such analogues may, for example, comprise the amino acid sequence of a naturally-occurring E. coli Ter binding polypeptide with one or more non io naturally-occurring amino acid substituents therein. Derivatives of a Ter binding polypeptide may include any functionally-equivalent fragments of the E. coli Ter binding protein or a homologue or analogue thereof having Ter binding activity, and any fusion polypeptides comprising E. coli Ter binding polypeptide or a homologue or analogue thereof and another protein wherein said fusion polypeptide has Ter binding activity. Ter binding polypeptide derivatives may include a fusion polypeptide comprising Tus and a polypeptide to be displayed on a microwell or microarray surface or on the surface of a cell, phage, virus or in vitro. Derivatives of a Ter binding polypeptide made be produced by chemical modification such as biotinylation or other suitable chemical modifications that a person skilled in the art would find suitable for the invention. Derivatives of a Ter binding polypetide can be made for the purpose of covalently crosslinking the derivaties to DNA.

As used herein, the term “Ter-binding activity” means the ability to bind to a naturally-occurring Ter site or to the double-stranded oligonucleotide of the present invention. Means for testing Ter-binding activity are described in the examples.

As used herein, the term “proteinaceous” shall be taken to include a cell, virus particle, bacteriophage, ribosome, polypeptide or a polypeptide fragment or a synthetic peptide.

As used herein, the term “complex” which can be used interchangeably with the term “conjugate” shall be taken to mean the binding of one molecule to one or more molecules. For example, a fusion protein may form a complex with DNA. As another example, a fusion protein may form a complex with a target molecule. In yet another example, a fusion protein may form a complex with DNA and a target molecule. As used herein, the term “conjugate” shall be taken to mean a composition of matter wherein one molecule is covalently attached or produced integrally with a second molecule. For example, a strand of the oligonucleotide of the present invention may be synthesized as a DNA/RNA hybrid molecule to integrate an mRNA molecule. Similarly, the strands of the double-stranded oligonucleotide may be synthesized to comprise additional sequence of a double-stranded oligonucleotide. In another alternative, a nucleic acid (DNA or RNA), polypeptide (e.g., a puromycin conjugate) or small molecule (e.g., a psoralen or derivative thereof) may be added post-synthetically to the double-stranded oligonucleotide by any conventional means known in the art.

As used herein, the term “in frame fusion” means that the nucleic acid encoding the Ter binding polypeptide with Ter binding activity and the nucleic acid encoding the peptide, polypeptide or protein of interest are in the same reading frame. Accordingly, transcription and translation of the nucleic acid results in expression of a single protein comprising both the Ter binding polypeptide with Ter binding activity and the peptide, polypeptide or protein of interest.

As used herein, the term “expression construct” shall be taken to mean a nucleic acid molecule that has the ability to confer expression of a nucleic acid fragment to which it is operably connected, in a cell or in a cell free expression system. Within the context of the present invention, it is to be understood that an expression vector that comprises a promoter as defined herein may be a plasmid, bacteriophage, phagemid, cosmid, virus sub-genomic or genomic fragment, or other Is nucleic acid capable of maintaining and or replicating heterologous DNA in an expressible format should it be introduced into a cell. Many expression vectors are commercially available for expression in a variety of cells. Selection of appropriate vectors is within the knowledge of those having skill in the art. The present invention contemplates an expression vector comprising a nucleic acid encoding a fusion protein of the invention.

As used herein, the term “promoter” is to be taken in its broadest context and includes the transcriptional regulatory sequences of a genomic gene, including the TATA box or initiator element, which may be required for accurate transcription initiation, with or without additional regulatory elements (ie. upstream activating sequences, transcription factor binding sites, enhancers and silencers) which alter gene expression in response to developmental and/or external stimuli, or in a tissue specific manner. In the present context, the term “promoter” is also used to describe a recombinant, synthetic or fusion molecule, or derivative which confers, activates or enhances the expression of a nucleic acid molecule to which it is operably linked, and which encodes the peptide or protein. Preferred promoters may contain additional copies of one or more specific regulatory elements to further enhance expression and/or alter the spatial expression and/or temporal expression of said nucleic acid molecule. These promoters may be phage T7 or lambda promoters.

As used herein, the term “partial or complete translation” shall be taken to mean that sufficient translation of mRNA occurs to produce a nascent polypeptide encoded by the mRNA to be detected e.g., by virtue of its activity or binding to a ligand (for example, a small molecule, antibody, protein binding partner, DNA recognition site, receptor, etc). As will be known to the skilled artisan, translation of a full-length polypeptide is not essential for such detection, and for most applications a polypeptide of at least 5-10 amino acids in length is generally sufficient.

As used herein, the term “conditions sufficient for partial or complete translation” means incubation of the mRNA conjugate in the presence of sufficient components of a suitable in vitro translation system e.g., wheat germ, reticulocyte lysate, or S-30 translation system. Commercially-available translation systems can be used. The methods disclosed herein are not limited to presentation or display involving eukaryotic mRNAs, as prokaryotic mRNAs are also contemplated. Accordingly, the in vitro translation system may be suitable for the translation of eukaryotic mRNA, on eukaryotic 80S ribosomes, or alternatively for the translation of prokaryotic mRNAs on 70S ribosomes.

As used herein, the term “nascent polypeptide” means a growing polypeptide chain produced by translation. In the present context, the term “nascent polypeptide” may be, but is not necessarily limited to, that part of a growing polypeptide chain exiting the ribosome.

As used herein, the term “chip” includes an array or microarray of any description, and includes a surface plasmon resonance chip, or “Biacore” chip.

As used herein, the term “monoplex format” shall be taken to mean a technique used for the detection and/or quantification of a single molecule in a single mixture or reaction wherein the mixture or reaction can be present in a well. For example, the term “monoplex format” is schematically defined in FIG. 1.

As used herein, the term “multiplex format” shall be taken to mean a technique used to detect and/or quantify two or more molecules wherein the molecules can be considered by a person skilled in art as being different and wherein these molecules can be detected and/or quantified in a single mixture or reaction wherein the mixture or reaction can be present in a well. For example, the term “multiplex format” is schematically defined in FIG. 2.

As used herein, the term “Barcode DNA” refers to a polynucleotide which comprises the TT Lock sequence as described herein and additional sequences which flank the 7 Lock sequence wherein the additional sequences assist in amplifying the TT Lock sequence by PCR. The Barcode DNA may contain a sequence which is approximately 10 to 90 base pairs in length, preferably 20 to 80 base pairs in length and more preferably 30 to 70 base pairs in length although any length of sequence is contemplated herein which allows the invention to be worked.

As used herein, the term “anti-target molecule” refers to any molecule that has an affinity or a specificity for a target molecule described herein. For example, the anti-target molecule may be an antigen or antibody.

As used herein, the term “target molecule” refers to any molecule which is to be detected, identified, amplified and/or quantified or any combination thereof. For example, the target molecule may be an antibody or an enzyme. The target molecule may be a biomarker wherein the biomarker is an indicator that a condition or disease is either present or predicted. For example, a biomarker could be prostate-specific antigen (PSA) for the detection of prostate cancer. The biomarker can be used as part of a screening method for the detection or prediction of a condition and/or disease .Throughout this specification, unless specifically stated otherwise or the context requires otherwise, reference to a single step, composition of matter, group of steps or group of compositions of matter shall be taken to encompass one and a plurality (i.e. one or more) io of those steps and in any order, compositions of matter, groups of steps or group of compositions of matter.

### BRIEF DESCRIPTION OF THE DRAWINGS

FIG. 1: Ultrasensitive PSA diagnostic. (A) Fusion protein is mixed with the Barcode DNA (DNA). (B) Serum containing PSA is mixed and incubated with DNA/fusion protein, and subsequently (C) transferred and incubated in an anti-PSA-coated 96-well plate to achieve the formation of the DNA/fusion protein/PSA/anti-PSA complex. (D) Subsequently, the PSA is detected by the detection of the DNA.

FIG. 2: Diagnostic application in multiplex format. A. A specific DNA molecule is cross-linked with a fusion protein to form a complex. B. Different target molecules are immobilized onto a surface through their interaction with specific capture antibodies (Abs). Each specific DNA/fusion protein complex binds to its respective target molecule. After several wash steps the signals are amplified by real-time PCR using sequence-specific Taqman probes.

FIG. 3: Crystal structures of the TuslTer variant complex (Mulcair et al., Cell 125, 1309-1319, 2006). (A). DNA is shown in cyan. Tus is represented in cartoon form. (B) Detail of site specific interactions. Note the stacking interaction between A(7) of Ter and F140 of Tus.

FIG. 4: Cloning strategy in pETMCSI backbone. A human c-myc 9E10 epitope (amino acid sequence EQKLISEEDLN) is N-terminally fused to a C-terminally His6 tagged soluble protein and cloned in a 17-promoter vector pETMCSI (Neylon et al., Biochemistry 39:11989-11999, 2000). The His6 tag is used to immobilize the 9E10 epitope using an anti His6 capture antibody. An E. coli codon optimized version of the gene encoding the anti-c-myc 9E10 scFv with Ndel-Ncol cloning sites, a pelB leader sequence at the N-terminus and a His6 tag at the C-terminus followed by an LPETG tag is custom synthesized and cloned alone or as fusion gene in-frame downstream or upstream of the tus gene. A soluble fusion protein or conjugate is produced and consist of Tus and the recombinant antibody fragment scFv 9E10 that binds specifically to the c-myc 9E10 epitope from expression in the periplasmic space of E. coli of various fusion genes, consisting of the pelB secretion signal, the scFv 9E10, a flexible linker sequence, and a C-terminally His6-tagged Tus, under the control of the T7 promoter. The N-terminal PeIB sequence directs the protein into the periplasm. The C-terminal His6 tag is followed by the sortase recognition—LPETG sequence. The construct with the scFv and Tus sequence in reverse order (see (C) and (D)) is expressed.

FIG. 5: Production of fusion proteins consisting of a scFv and Tus. The fusion proteins are purified using Ni-NTA affinity chromatography. The optimal position of Tus (N- or C-terminus) in the fusion protein and what is the optimal size and composition of the flexible linker (GGGS)n separating the two domains is investigated.

FIG. 6: Sortase catalyzed ligation of Tus with scFv. For efficient ligation of the two proteins, the enzyme sortase is used. A sequence coding for an N-terminal GGG—tag is fused in frame with the tus gene and cloned in pETMCSI. The GGG-Tus is expressed and purified by Ni-NTA affinity chromatography. The ligation of purified Tus and the scFv 9E10 is then carried out analogously to the method described by Mao et al., J Am Chem Soc 126:2670-2671, 2004).

FIG. 7: Study of a protein complex. (A) A protein complex is pulled down by immunoprecipitation. (B) The complex is analyzed with various specific DNA/Tus-anti-target conjugates.

FIG. 8: PCR profile. Table which shows the cycling conditions such as number of cycles and temperatures used for each cycle of real-time PCR.

FIG. 9: Raw data from real-time PCR. Fluorescence from three standards (i.e. 1nM, 10 pM and 100 fM) and three samples containing anti GFP antibody (at a concentration of 0 pg, 1 pg or 100 pg) and a negative control (i.e. no template) was detected by real time PCR and plotted. FIG. 10: Normalized data from real-time PCR. Fluorescence from three Barcode DNA standards (i.e. 1 nM, 10 pM and 100 fM) and three samples containing anti GFP antibody (0 pg, 1 pg and 100 pg) and a negative control (i.e. no template) was detected and quantififed by real time PCR.

FIG. 11: Standard curve for real-time PCR. A standard curve for real time PCR was generated from three standards (i.e. 1 nM, 10 pM and 100 fM) of Barcode DNA. Real-time PCR results of three samples containing anti GFP antibody (at a concentration of 0 pg, 1 pg or 100 pg) were than plotted onto the standard curve for comparison to determine the background and limit of detection of the assay.

FIG. 12: Results table. Table which depicts the comparison between the amount of anti GFP antibody (0 pg, 1 pg or100 pg) present in a sample as assessed by real-time PCR and the standard curve reflecting maximal theoretical binding values. This table depicts the overall efficiency of the system translated by the percent of total binding in the time frame of the assay.

FIG. 13: UV Cross-linking. Table which depicts the percent of cross-linking between a Ter binding protein and a Ter derivative.

FIG. 14: Qualitative assessment of UV-cross-linking. A droplet comprising 3 p L of Ter-binding protein and 3 p L of annealed oligonucleotides is deposited in a 12 well multidish (Nunclon) and left at room temperature for 10 minutes. The 12 well multidish is turned upside down without lid over a transilluminator and irradiated at 312 nm during 5 minutes. A pre-chilled aluminium block (−20 C) is positioned over the dish to avoid overheating. The yield of crosslinking was assessed by SDS-PAGE electrophoresis using a 12.5% nextgel (Amresco).

### DETAILED DESCRIPTION

The development of molecular diagnostics to ensure positive patient outcome is central to modern medical diagnosis to enable detection of life-threatening diseases at an early stage when they can be effectively treated.

While the inventors' ultrasensitive diagnostic system described herein specifically targets detection of prostate cancer recurrence after surgery or radiation therapy a person skilled in the art would consider the technology amenable to detection of other cancers and infectious or neurodegenerative diseases, e.g. ovarian, breast, lung or colon cancer, HIV and Alzheimer's and Parkinson's disease. The new ultrasensitive diagnostic system is expected to be more sensitive than currently available tests. In particular for PSA this is very important to ensure that after radical prostatectomy, all of the prostatic tissue has been removed and that there are no cancerous cells left. A more sensitive test will also mean that, if necessary, post surgery chemotherapy could be started earlier with a better prognosis.

The assay described herein is simple and does not require expensive chemistry or purification steps. It will ultimately require a standard laboratory setup without the need of special and expensive instrumentation. Due to its size and particular design, the Barcode DNA will not interfere with the different steps of the assay.

For example, the PSA assay described herein serves as the foundation and proof of concept for the use of the TT-Lock based untrasensitive signal amplification system (USAS) to develop new ultrasensitive diagnostics directed towards biomarkers present at very low concentrations in body fluids or other specimens. It is anticipated that this technology could simply be modified for the detection of viral antigens like the HIV p24 protein. Detection of bacterial contamination in water or food is also envisaged.

Currently, the two main methods used to link DNA to antibodies are as follows: (1) Self-assembling biotinylated DNA/streptavidin technology and (2) Chemical cross-linking method. In both cases a mixture of products is generated and expensive purification and labeling chemistries are necessary to generate the reagents. It is also obvious that discrepancies between batches are expected with these methods due to the non-stoichiometric nature of the process. Streptavidin is a tetrameric protein and chemical crosslinking can bind more than one DNA molecule or alter the binding properties of the antibody to the biomarker.

### Advantages of the TT-Lock DNA

The TT-Lock DNA is a partially forked 21-bp DNA of specific sequence that makes an extremely stable interaction with Tus, a monomeric DNA-binding protein from E. coli (Mulcair et al., Cell 125, 1309-1319, 2006). This protein-DNA interaction is the most stable reported for a monomeric protein binding to DNA, and methods are described herein which show the use of the interaction of Tus and the TT-Lock for use in multiplex assay format. The TT-Lock offers a new and easy way to link DNA to an antitarget molecule for the reliable and sensitive amplification of signal using routine DNA amplification and fluorescence methods. As such, this very strong protein-DNA interaction has biotechnological applications in situations where DNA or antibodies are immobilized on surfaces, and has potential especially to solve existing problems with diagnostic applications based on highly sensitive ImmunoPCR methods.

### Signal Generating Systems

Within the scope of the present invention is the means to achieve the ultrasensitive detection of a target protein in a reproducible and fast way. The most common signal generating systems are based on radioactive, chemiluminescent or fluorescent probes using chemical cross-linkers and are contemplated herein. The common problems are inactivation of the device and low yields. Fluorescent probes can be used that will bind specifically to a DNA recognition sequence corresponding to each respective target. The multiplex possibilities of these new technologies are only limited by the specifications of the instrument (currently 5 different channels) used for detection (Molenkamp et al., J Virol Methods, 141:205-211, 2007). With the power of real-time PCR (Klein, Trends Mol Med 8:257-260, 2002) the detection limits achieved with these technologies are also greater, since single target molecules can potentially be detected. These advantages are particularly significant for the sheer number and stringency of diagnostic tests performed, for example by blood banks. The utility of this technique as contemplated herein is for any precise quantitative study of any given molecule and is not limited only to the detection of protein antigens.

The current methods used for the production of DNA-antibody hybrids and some of their associated problems have been discussed in Niemeyer et al., Trends Biotechnol 23:208-216 2005. One of the major issues is that the majority of the methods for coupling the DNA to the antibody use the streptavidin (SA)-biotin interaction (Weber et al., Science 243:85-88,1989). The manufacture of biotinylated DNA is expensive and the downstream use of technologies based on the SA-biotin interaction is prohibited due to potential cross-reactivity. To avoid some of these issues, a new method which is contemplated within the present invention has been recently developed that makes use of intein-based expressed protein ligation to generate the so-called tadpoles (Lovrinovic et al., J Chem Soc Chem Commun, 822-823, 2003; Burbulis et al., Nat Methods, 2:31-37, 2005). These methods eliminate potential heterogeneity in the antibody- DNA hybrid, but although they are very elegant, another dimension of complexity is introduced by the complexity of the chemistry involved, and they will be very expensive to use in practice. Some of the signal generation systems used herein and contemplated in this invention include but are not limited to flourescent, PCR, radioactive and intercalating dye based systems. Signal generation systems contemplated herein include but are not limited to systems that detect, identify, screen, and quantify one or more target molecules. Furthermore, the signal generation system may or may not require amplification of one or more target molecules. A person skilled in the art would understand that a signal generation system used to perform the invention is not limited to the signal generation systems described and exemplified herein.

### Oligonucleotide Synthesis of Barcode DNA

The oligonucleotides for use in the present invention may be produced by recombinant or chemical means known to the skilled artisan. The oligonucleotides for use in the present invention may be less than about 100 nucleotides in length, and in particular may be no more than about 30 or 35 or 40 or 45 or 50 or 60 or 70 nucleotides in length, and may not comprise completely complementary first and second strands, chemical synthesis of each strand separately, followed by annealing of the first and second strands under appropriate hybridization conditions may be preferred.

DNA of up to about 80 nucleotides in length may be conveniently synthesized by chemical means. Longer molecules may generally be manufactured by amplification using PCR directly from template DNA by annealing overlapping oligonucleotide primers and primer extension of the overlapping ends to produce a full-length double-stranded nucleic acid molecule, for example, as described by Stemmer et al., Gene 164:49-53, 1995; Casimiro et al., Structure 5:1407-1412, 1997.

The solid phase chemical synthesis of DNA fragments may be routinely performed using protected nucleoside phosphoramidites, for example, as described by Beaucage et al., Tetrahedron Lett 22:1859, 1981. In general, the 3′-hydroxyl group of an initial 5′-protected s nucleoside may be covalently attached to a polymer resin support, for example, as described by Pless et al., Nucleic Acids Res 2:773, 1975. Synthesis of the oligonucleotide may then proceed by deprotection of the 5′-hydroxyl group of the attached nucleoside, followed by coupling of an incoming nucleoside-3′-phosphoramidite to the deprotected hydroxyl group, for example, as described by Matteucci et al., J Am Chem Soc 103:3185, 1981. The resulting phosphite triester may be oxidized to a phosphorotriester to complete the internucleotide bond (see, for example,

Letsinger et al., J Am Chem Soc 98:3655, 1976). The steps of deprotection, coupling and oxidation may be repeated until an oligonucleotide of the desired length and sequence is obtained.

The chemical group conventionally used for the protection of nucleoside 5′-hydroxyls may be dimethoxytrityl (“DMT”), which is removable using acid (Khorana, Pure Appl Chem 17:349, 1968; Smith et al., J Am Chem Soc 84:430, 1962) and may aid separation on reverse-phase HPLC (Becker et al., J Chromatogr 326:219, 1985). Alternatively, 5′-O-protecting groups which may be removed under non-acidic conditions may be used, for example, as described by Letsinger et al., J Am Chem Soc 89:7147, 1967; Iwai et al., Tetrahedron Lett 29:5383, 1988; Iwai et al., Nucleic Acids Res 16:9443, 1988. Seliger et al., Nucleosides & Nucleotides 4:153, 1985 also describe a 5′-O-phenyl-azophenyl carbonyl (“PAPco”) group, which may be removed by a two-step procedure involving trans-esterification followed by beta-elimination. Fukuda et al., Nucleic Acids Res Symposium Ser 19:13, 1988, and Lehmann et al., Nucleic Acids Res 17:2389, 1989 also describe application of a 9-fluorenylmethylcarbonate (“Fmoc”) group for 5′-protection which produces yields for the synthesis of oligonucleotides up to 20 nucleotides in length. Letsinger et al., J. Am. Chem. Soc. 32, 296 (1967) also describe the use of a p-nitrophenyloxycarbonyl group for 5′-hydroxyl protection. Dellinger et al., US Patent Publication No. 20040230052 (18 November 2004) also describe rapid and selective deprotection of 5′-OH or 3′-OH nucleoside carbonate groups using peroxy anions in aqueous solution, at neutral or mild pH.

Means for chemically synthesizing RNA are described, for example, in US Patent Publication No. 0040242530 (2 Dec. 2004) which is incorporated herein in its entirety. These methods rely upon 5′-DMT-2′-t-butyldimethylsilyl (TBDMS) or 5′-DMT-2′-(2-fluorophenyl)-4-methoxypiperidin-4-yl] (FPMP) chemistries that are readily available commercially.

In summary, nucleosides may be suitably protected and functionalized for use in solid-phase or solution-phase synthesis of RNA oligonucleotides. For example,. syntheses may be performed on derivatized polymer supports using either a Gene Assembler Plus synthesizer (Pharmacia) or a 380B synthesizer (ABI). A 2′-hydroxyl group in a ribonucleotide may be modified using a Tris orthoester reagent, to yield a 2-O-orthoester nucleoside, by reacting the ribonucleoside with the tris orthoester reagent in the presence of an acidic catalyst, for example, pyridinium p-toluene sulfonate. The product may then be subjected to protecting group reactions (e.g., 5′-O-silylation) and functionalizations (e.g., 3′-O-phosphitylation) to produce a nucleoside phosphoramidite for incorporation within an oligonucleotide or polymer by reactions known to those skilled in the art. Following synthesis, the polymer support may be treated to cleave the protecting groups from the phosphates (including base-labile protecting groups) and to release the 2′-protected RNA oligonucleotide into solution. Crude reaction mixtures may then be analyzed by anion exchange high pressure liquid chromatography (HPLC) and subjected to sequence analysis. RNA may also be produced by in vitro transcription of DNA encoding each strand of a double-stranded oligonucleotide of the invention, for example, by being cloned into a plasmid vector or an oligonucleotide template using an RNA polymerase enzyme, for example, E. coli RNA polymerase, bacteriophage SP6, T3, T7 RNA polymerase, an error-prone RNA polymerase such as Oβ-replicase or other viral polymerase. In vitro methods for synthesizing single stranded RNAs of defined length and sequence using RNA polymerase are described by Milligan et al., Nucleic Acid Res 15:8783-8798, 1987 and in US Patent Publication No. 20040259097 (23 Dec. 2004).

For the production of double-stranded RNA using an RNA polymerase, both a sense and an antisense oligonucleotide template may be required to be separately transcribed and the reaction products annealed. The oligonucleotide templates may be synthetic DNA templates or templates generated as linearized plasmid DNA from a target-specific sequence cloned into a restriction site of a vector such as for example a prokaryotic cloning vector (pUC13, pUC19) or PCR cloning systems such as the TOPO cloning system of Invitrogen. Synthetic DNA templates may be produced according to techniques well known in the art.

An RNA polymerase enzyme may form an RNA polymer from ribonucleoside 5′-triphosphates that is complementary to the DNA template. The enzyme may add mononucleotide units to the 3′-hydroxyl ends of the RNA chain and thus build RNA in the 5′-to-3′ direction, antiparallel to the DNA strand used as template. DNA-dependent RNA polymerases such as E. coli RNA polymerase, RNA-directed RNA polymerases such as the bacteriophage RNA polymerases (i.e., RNA replicases), or bacterial polynucleotide phosphorylases may be used in this context.

RNA polymerases generally require the presence of a specific initiation site or RNA polymerase promoter sequence within each DNA template to bind the RNA polymerase and initiate transcription. A minimum or truncated RNA polymerase promoter sequence, wherein one or more nucleotides of a naturally-occurring promoter sequence are deleted may also be employed, with no or little effect on the binding of the RNA polymerase to the initiation site and with no or little effect on the transcription reaction.

The reaction conditions for transcription reactions performed in vitro are known in the art to comprise a DNA template, an RNA polymerase enzyme and the nucleoside triphosphates (NTPs) for the four required ribonucleotide bases, adenine, cytosine, guanine and uracil, in a reaction buffer optimal for the RNA polymerase enzyme activity. For example, the reaction mixture for an in vitro transcription using T7 RNA polymerase typically contains, T7 RNA polymerase (0.05 mg/ml), oligonucleotide templates (1 p M), each NTP (4 mM), and MgCl₂ (25 mM) which supplies Mg²+ as a co-factor for the polymerase. This mixture may be incubated at about 37° C. in a buffer comprising 10 mM Tris-HCI pH 8.1 for several hours (see Milligan & Uhienbeck, Methods Enzymol 180:51-62, 1989). Such reagents are commercially available e.g., MEGA shortscript T7 kit (Ambion).

The oligoribonucleotide transcription products may be purified by any method known in the art such as, for example, gel electrophoresis, size exclusion chromatography, capillary electrophoresis or HPLC. Gel electrophoresis may be typically used to purify the full-length transcripts from the reaction mixture, but this technique may not be amenable to production on a large scale. Size exclusion chromatography, such as using Sephadex G-25 resin (Pharmacia), optionally combined with a phenol:chloroform:isoamyl alcohol extraction and ethanol precipitation may be more appropriate for large scale preparations.

To obtain double-stranded DNA (dsDNA) or double-stranded RNA (dsRNA) or a double-stranded hybrid molecule such as an RNA/DNA hybrid, the two strands may be annealed by standard means known to the skilled artisan. For example, the first and second strands may be brought into contact with each other at a temperature below their predicted Tm and/or in a medium comprising a salt such as KCI, MgCl₂ or NaCI.

TT-Lock Oligonucleotide Structure

The foregoing modifications may or may not produce a forked structure downstream of a cytosine residue of the second strand that is conserved in a naturally-occurring Ter site and involved in fork arrest. Alternatively, a modification that produces a forked structure in the double-stranded oligonucleotides of the present invention may occur upstream of a naturally-occurring guanosine residue in the first strand in a naturally-occurring Ter site. If such an upstream forked structure is present, base-pairing with the other strand through this modified nucleotide residue may not occur in the double-stranded oligonucleotides. A modification that produces a forked structure in the double-stranded nucleic acid molecule may include modification of this guanosine residue on the first strand, and in particular may include one or two or three nucleotide residues downstream of the guanosine residue in the first strand. The fork may be any length, and may comprise 1-5 or 5-10 or 10-15 or 15-20 nucleotides in length. The length of this fork may modify the rate of dissociation of the double-stranded oligonucleotide from a Ter binding polypeptide, such that dissociation rates may become progressively faster as the length of the fork increases, with or without simultaneous mutation of the other strand. For example, forks produced by the addition of up to about five nucleotide residues from a naturally-occurring TerB site to the first strand sequence of the oligonucleotides as described above may exhibit half-lives for dissociation from Tus at 20° C. that are at least approximately the same as for a wild-type TerB oligonucleotide. Similarly, forks that are produced by the addition of up to about four nucleotide residues from a naturally-occurring TerB site to the second strand is sequence of the oligonucleotides as described above may exhibit half-lives for dissociation from

Tus at 20° C. that are at least approximately the same as for a wild-type TerB oligonucleotide. The subsequent mutation of such forks by substitution of up to about four of these additional nucleotides in the 5′-region of the first strand or the second strand may not reduce the half-life for dissociation from Tus relative to the wild-type TerB sequence. In contrast, a fork-producing mutation, for example a substitution or deletion, of five or more nucleotides positioned upstream of the central core sequence 5′- GTTGTAAC-3′ (SEQ ID NO: 3) in the first strand of native TerB, may increase the half-live of dissociation of the double-stranded oligonucleotide from a Ter binding polypeptide by at least about 10-fold, at least about 20-fold or at least about 50-fold relative to a wild-type TerB. Such mutations may also be combined with one or more nucleotide mutations, for example, substitutions downstream of the conserved cytosine involved in fork arrest of native TerB sites without adversely affecting half-life of Ter/Tus complex formation. It will be appreciated by the skilled artisan that a higher half-life for dissociation of the double-stranded oligonucleotide from a Ter binding polypeptide may be desirable for display or presentation of a molecule using the interaction between the oligonucleotide and a Ter binding polypeptide. This is because complexes that dissociate rapidly may be too unstable to permit operations to be performed.

The conserved cytosine residue involved in fork arrest of a naturally-occurring Ter site (e.g, native TerB) may not be base-paired in the double-stranded oligonucleotide of the present invention, especially when it comprises a fork structure positioned upstream of the central core sequence 5′- GTTGTAAC-3′ (SEQ ID NO: 3) in the first strand. Mispairing of this residue exhibits very slow dissociation rates (that is, a “locked” behaviour) and is particularly suitable for displaying or presenting any molecule.

Forked structures may be conveniently produced by synthesizing first and second strand oligonucleotides and annealing the strands, wherein the sequence upstream of the central core sequence 5′- GTTGTAAC-3′ (SEQ ID NO: 3) in the first strand may be non-complementary to a sequence downstream of a complementary central core sequence (for example in the 3′-region) of the second strand.

Alternatively, an open loop may be included upstream or downstream from the central core sequence without adversely affecting the half-life for dissociation of the double-stranded oligonucleotide from a Ter binding polypeptide. Such loops may comprise one or two or three or four or five or more consecutive residues. The loop may comprise and/or flank a conserved cytosine residue involved in fork arrest. A loop may be introduced into the double-stranded oligonucleotides of the invention by introducing one or more nucleotide substitutions into the first and/or second strand sequence of a naturally-occurring Ter site. For example, a loop may be is produced by synthesizing first and second strand oligonucleotides and annealing the strands, wherein the upstream sequence proximal to the central core sequence 5′- GTTGTAAC-3′ (SEQ ID NO: 3) in the first strand is non-complementary to a sequence in the second strand and the upstream sequence distal thereto is complementary to a 3′-region of the second strand sequence.

### Conjugation of an Oligonucleotide to a Polypeptide or Protein

In one embodiment, the oligonucleotides for use in the invention or a first or second strand thereof may be conjugated to another molecule of interest such as a peptide, polypeptide, protein, antibody or antibody fragment.

The oligonucleotides may be derivatized to include 5′- and/or 3′- insertions that do not adversely affect its ability to bind to a Ter binding polypeptide. The insertions may include the addition of mRNA and/or DNA that is to be presented or displayed.

In another embodiment, the oligonucleotides as described above may be bound to one or more proteinaceous molecules, nucleic acid molecules, or small molecules. The binding may be covalent or non-covalent. Non-covalent binding of the oligonucleotides may be to a Ter binding polypeptide (e.g., SEQ ID NO; 5) having TerB-binding activity such as, for example, a fusion polypeptide comprising Tus and a polypeptide to be displayed on a microwell or microarray surface or on the surface of a cell, phage, virus or in vitro. Covalent linkages may be between the double-stranded oligonucleotides and a non-Ter binding proteinaceous molecule, nucleic acid molecule, or small molecule.

In a further embodiment, the oligonucleotide as described above may be bound to:

(i) a Ter binding polypeptide (e.g., SEQ ID NO; 5) having TerB-binding activity; and

(ii) a proteinaceous molecule, nucleic acid molecule, or small molecule.

The oligonucleotide derivative may therefore further comprise DNA or RNA to be displayed on a microwell or microarray surface or on the surface of a cell, phage, virus or in vitro. The Ter binding polypeptide derivative may be a fusion polypeptide comprising Tus and a polypeptide to be displayed on a microwell or microarray surface or on the surface of a cell, phage, virus or in vitro.

It will also be apparent from the disclosure herein that the oligonucleotides for use in the io present invention may be particularly useful for presenting or displaying one or more other molecules to which it can be conjugated or covalently attached during synthesis or post-synthesis.

Accordingly, the present invention also provides a complex comprising the oligonucleotides as described herein and another molecule, for example, a nucleic acid, polypeptide or small molecule.

In a further embodiment, the oligonucleotides bound as described above are used for presentation or display. For example, a Ter binding polypeptide, fragment or derivative thereof having TerB binding activity may be conjugated to a peptide, polypeptide, antibody or fragment thereof, or a small molecule, and presented in combination with the double-stranded oligonucleotide for assay purposes. As will be known to the skilled artisan, the peptide, polypeptide or antibody fragment may be produced by recombinant means as an in-frame fusion with a Ter binding polypeptide. Alternatively, a peptide, polypeptide, antibody or fragment thereof, or a small molecule may be conjugated to a Ter binding polypeptide by chemical means. Accordingly, the present invention also encompasses a complex comprising a Ter binding polypeptide and another molecule. The conjugate may be a Ter binding polypeptide derivative.

It is also within the scope of the present invention to use a conjugate comprising mRNA encoding a Ter binding polypeptide fused in the same reading frame to mRNA encoding a second polypeptide.

Methods for conjugating a nucleic acid to a peptide, polypeptide or protein are known in the art and include, for example, covalent or non-covalent conjugation. For example, a non-covalent interaction, such as an ionic bond, a hydrophobic interaction, a hydrogen bond and/or a van der Waals attraction may be used to produce a nucleic acid:protein conjugate. Such a non-covalent interaction may be produced, for example, using an ionic interaction involving a modified nucleic acid and residues within the peptide, polypeptide or protein, such as charged amino acids, or by using of a linker comprising charged residues that interacts with both the nucleic acid and the peptide, polypeptide or protein. For example, non-covalent conjugation may occur between a generally negatively-charged modified nucleic acid and positively-charged amino acid residues of a peptide, polypeptide or protein, for example, polylysine and/or polyarginine residues.

Alternatively, a non-covalent conjugation between a nucleic acid and a peptide, polypeptide or protein may be produced using a DNA binding motif of a molecule that interacts with nucleic acid as a natural ligand. For example, such DNA binding motifs may be found in transcription factors and anti-DNA antibodies. By fusing the nucleic acid to the binding site of the DNA binding motif, and the peptide, polypeptide or protein to the DNA binding motif a non-covalent interaction may be produced.

In another embodiment, a covalent interaction may used to produce a nucleic acid:protein conjugate. A general method to form a protein:nucleic acid conjugate involves coupling a linker compound to an oligonucleotide sequence during synthesis. If necessary a functional group on the linker and/or on the oligonucleotide may then be deprotected, for example, by ammonia or hydroxide treatment. A suitable method of deprotection will be apparent to the skilled artisan. The linker may then be activated and the modified oligonucleotide reacted with a peptide, polypeptide or protein to form a covalent linkage. Suitable examples of this method are described, for example, in Agrawal et al., Nucleic Acids Res 14:6227-6245, 1986 or Connolly Nucleic Acids Res 13:4485- 4502, 1985; or U.S. Pat. Nos. 4,849,513; 5,015,733; 5,118,800; and 5,118,802.

In a specific example of this method, a linker containing a carbomethoxy group may be coupled to a resin-bound oligonucleotide in a DNA synthesizer. After simultaneous deprotection (should the oligonucleotide contain any protecting groups), ester hydrolysis and resin removal, the newly formed carboxylic acid may be activated with a carbodiimide, such as, for example, 1-ethyl-3-(dimethylaminopropylcarbodiimide) (EDAC), N-hydroxysuccinimide, N-hydroxybenzotriazole, or tetrafluorophenol may be added to form an active ester in situ. This activated carboxyl group may then be reacted with a peptide, polypeptide or protein to form a covalent oligonucleotide-linking group-peptide, -polypeptide or -protein conjugate.

In another example, Zuckermann et al., Nucleic Acids Res 15:5305-5321, 1987 describes a method for conjugating a peptide, polypeptide or protein to the 3′ end of a nucleic acid. The method involves the incorporation of a sulphydryl group into the 3′-nucleotide or nucleoside-support linkage as a disulfide bond, prior to automated oligonucleotide synthesis. The approach described avoids complications due to functionalities present in the final oligonucleotide. The oligonucleotide may be synthesized from the thiolated 3′-terminal nucleoside (or nucleotide) using standard solid phase phosphotriester or phosphoramidite chemistry, deprotected by conventional methods, treated with dithiothreitol (DTT), and purified by reverse phase chromatography. The thiolated oligonucleotide may then be activated with 2,2′-dithiodipyridine and cross-linked to a thiol containing peptide, polypeptide or protein. Alternatively, the 3′-thiol-containing oligonucleotide may be derivatized with an electrophile such as an N-haloacetyl or maleimidyl group conjugated to the peptide, polypeptide or protein.

Alternatively, a peptide, polypeptide or protein may be conjugated to the 3′-end of a nucleic acid through solid support chemistry. For example, the nucleic acid may be added to a polypeptide portion that has been pre-synthesized on a support as described in Haralambidis et al., Nucleic Acids Res 18:493-499, 1990 or Haralambidis et al., Nucleic Acids Res 18:501-505, 1990. These methods may involve the synthesis of a peptide or polypeptide of interest on a solid support, for example, using Boc chemistry. At the terminus of the peptide or polypeptide polyamide, synthesis may be performed and the terminal amino group converted to a protected primary aliphatic hydroxy group by reaction with alpha, omega-hydroxycarboxylic acid derivatives. Oligonucleotide synthesis may then be performed using phosphoramidite chemistry

In another embodiment, the nucleic acid may be synthesized such that it is connected to a solid support through a cleavable linker (a modified nucleic acid) extending from the 3′ terminus. Upon chemical cleavage of the modified nucleic acid from the support, a terminal thiol group may be left at the 3′-end of the oligonucleotide (Corey et al., Science 238:1401-1403, 1987) or a terminal amine group left at the 3′-end of the oligonucleotide (Nelson et al., Nucleic Acids Res 17:1781-1794, 1989). Conjugation of the amino-modified nucleic acid to amino groups of a peptide, polypeptide or protein may then be performed as described in Benoit et al., Neuromethods 6:43-72, 1987. Conjugation of the thiol-modified modified oligonucleotide to carboxyl groups of the peptide may be performed as described in Sinah et al., Oligonucleotide Analogues. A Practical Approach, IRL Press, 1991.

Compounds may also be attached to the 3′ end of oligomers, as described by Asseline et al., Tet Lett 30:2521, 1989. This method utilizes 2,2′-dithioethanol attached to a solid support to displace diisopropylamine from a 3′ phosphonate bearing an acridine moiety that may be subsequently deleted after oxidation of the phosphorus. Other substituents have been bound to the 3′ end of oligomers by alternate methods, including the use of polylysine (Bayard et al., Biochemistry 25:3730, 1986). Additional methods of attaching non-nucleotide compounds to oligonucleotides are discussed in U.S. Pat. Nos. 5,321,131 and 5,414,077.

In another embodiment, the peptide, polypeptide or protein may be conjugated to the 5′ end of the oligonucleotides of the invention. For example, Haralambidis et al., Nucleic Acids Res 15:4857-4876, 1987 describe a method for conjugating a nucleic acid to a peptide, polypeptide or protein. This method utilises a C-5 substituted deoxyuridine nucleoside in the production of an oligonucleotide. The substituent carries a masked primary aliphatic amino group. This key intermediate may then be functionalized at its C-5 substituent to give nucleosides with longer C-5 arms. The resulting oligonucleotide may then readily be reacted with a peptide, polypeptide or protein of interest to produce a conjugate.

In another embodiment, a nucleic acid may be produced that is linked to a moiety comprising a free amine group. The amine may then be derivatized with a maleimide- or haloacetyl-containing heterobifunctional agent, such as N-succinimidyloxy-4(N-maleimido-methyl)- cyclohexane-1 carboxylate (SMCC) or iodoacetic anhydride, and then conjugated to a thiol group on a peptide, polypeptide or protein. Alternatively, the amine functional group may be reacted with succinic anhydride, with the resultant free carboxylic acid group subsequently being coupled to an amine group on the peptide, polypeptide or protein using carbodiimide.

In a further alternative embodiment, the amine functional group may be reacted with a thiol-containing heterobifunctional reagent, such as iminothiolane or succinimidyloxy-3-2 (2- pyridyldithio) propionate (SPDP), followed by a treatment with a reducing agent, such as -mercaptoethanol or dithiothreitol (DTT). The resultant free thiol group may be reacted with a maleimide or haloacetyl derivative of a peptide, polypeptide or protein. This derivatization of the peptide, polypeptide or protein may be accomplished, for example, via reaction with SMCC, iodoacetic anhydride or N-succinimidyloxy-(4-iodoacetyl) aminobenzoate (STAB) under neutral or slightly alkaline conditions.

In another embodiment, a disulfide-bonded conjugate may be produced using an unreduced SPDP-oligonucleotide derivative as described together with a thiol-containing peptide, polypeptide or protein. Should the peptide, polypeptide or protein not contain a native thiol, the peptide, polypeptide or protein may be derivatized with iminothiolane or SPDP, followed by reduction with DTT or 3-mercaptoethanol, or via DTT-mediated reduction of native disulfides.

Alternative methods for linking compounds, such as proteins, labels, small molecules, oligonucleotides and other chemical entities, to nucleotides are known in the art. For example, substituents may be attached to the 5′ end of a preconstructed oligonucleotide using amidite or H- phosphonate chemistry, as described by Ogilvie, et al., Pure Appl Chem 59:325, 1987, and by Froehler, Nucleic Acids Res 14:5399, 1986.

Ms/TT-Lock Complex

It is contemplated herein that the TusiTT-Lock (or Tus-GFP/TT-Lock or other Tus-fusion derivative/TT-Lock) complex does not interfere with other common chemistries used in immunoassays. This allows us to use the streptavidin-biotin interaction if necessary to immobilize the capture antibody in high yields with virtually no bleeding during washing steps. One embodiment contemplated herein to reduce non-specific interferences is to use only the binding domains of antibodies (Warren et al., Clin Chem 51:830-838, 2005), although this often leads to a reduction of affinity to the antigen. The production of antibody fusion proteins can be achieved, but requires elaborate protein engineering due to the heterotetrameric structure of antibodies. Affinity maturation of binding domains can be achieved through directed evolution of the variable domain of engineered single chain antibody fragments (scFv) and selection from phage or ribosome display libraries (Pavoni et al., BMC Cancer 6:41, 2006; Vaccaro et al., J Immunol Methods 310:149-158, 2006; Ohashi et al., Biochem Biophys Res Commun 352:270-276, 2007; Jermutus et al., Proc Natl Acad Sci USA 98:75-80, 2001). High affinity monoclonal antibodies with very slow off-rates are particularly useful in diagnostic applications, since once bound to their target antigens, they dissociate little during successive wash steps.

Therefore, the 7-Lock technology allows a higher degree of flexibility in the protocols compared to the other methods, since the addition of the self-assembling Barcode DNA can occur at any time during the incubation or wash steps to achieve the best detection conditions.

Conjugation of an Oligonucleotide to a Non-Proteinaceous Compound

In another embodiment, the oligonucleotides used in the present invention are conjugated to a non-proteinaceous molecule such as a lipid, oligosaccharide or small molecule. Several of the methods described above may be also useful for conjugating a nucleic acid of the invention to such non-proteinaceous compounds. For example, production of a nucleic acid linked to a moiety comprising a free amine group may facilitate the use of a chemical cross-linking agent that may be useful for linking the oligonucleotides to any of a variety of compounds.

An oligonucleotide for use in the invention may be linked to a lipid using a method known in the art, such as, for example, synthesis of oligonucleotide-phospholipid conjugates (Yanagawa et al., Nucleic Acids Symp Ser 19:189-192, 1988), oligonucleotide-fatty acid conjugates (Grabarek et al., Anal Biochem 185:131-135, 1990; and Staros et al., Anal. Biochem 156:220-222, 1986), and oligonucleotide-sterol conjugates (Boujrad et al., Proc Natl Acad Sci USA 90:5728-5731, 1993).

The linkage of a nucleic acid of the invention to an oligosaccharide may be achieved using a method, such as, for example, the synthesis of oligonucleotide-oligosaccharide conjugates, wherein the oligosaccharide may be a moiety of an immunoglobulin (as described in O′Shannessy et al., J Applied Biochem 7:347-355, 1985).

Conjugation of an Oligonucleotide to another Nucleic Acid

In yet another embodiment, the oligonucleotides for use in the invention are conjugated to a nucleic acid of interest. In this regard, the nucleic acid of interest may comprise DNA, RNA, a derivative of DNA, a derivative of RNA or a combination thereof. Furthermore, the nucleic acid of interest may be, for example, single stranded, duplex or triplex nucleic acid.

Methods for the production of such conjugated nucleic acids are known in the art and described, for example, in Ausubel et al. (In: Current Protocols in Molecular Biology, Wiley Interscience, ISBN 047 150338, 1987) and Sambrook et al. (In: Molecular Cloning: A Laboratory Manual, Cold Spring Harbor Laboratories, New York, Third Edition, 2001). For example, a nucleic io acid molecule comprising the nucleic acid of the invention and a nucleic acid of interest may be synthesized. Methods of oligonucleotide synthesis are known in the art and described, for example, in Gait (Ed) (In: Oligonucleotide Synthesis: A Practical Approach, IRL Press, Oxford, 1984). In this regard, the nucleic acid synthesized may comprise any combination of nucleotides (e.g., DNA or RNA) and/or nucleotide analogues or derivatives.

Alternatively, a single nucleic acid molecule comprising the oligonucleotides of the invention and a nucleic acid of interest may be produced using recombinant means, such as, for example, splice overlap extension. For example, an oligonucleotide of the invention may be amplified using, for example, PCR, in which one of the primers used in the reaction comprises a sequence that is capable of hybridizing to the nucleic acid of interest. By using the resulting amplification product in a further PCR reaction to amplify the nucleic acid of interest, a single nucleic acid molecule comprising both the oligonucleotide of the invention and the nucleic acid of interest may be produced.

The method of Tian et al., Nature 432: 1050-1054, 2004 may be particularly useful for synthesising long strands of nucleic acid. This method essentially involves synthesizing a plurality of oligonucleotides that span the sequence of the nucleic acid to be produced (for example, a nucleic acid of the invention linked to a nucleic acid of interest), wherein the oligonucleotides may be synthesised on a microchip. Each oligonucleotide may comprise a restriction endonuclease site to thereby facilitate its release from the microchip. By releasing the oligonucleotides from the chip and using them in a PCR reaction (i.e., splice overlap extension) a single nucleic acid molecule may be produced.

In a further embodiment, a conjugate comprising double stranded DNA or RNA or a double stranded DNA/RNA conjugate may be produced using a DNA ligase, such as, for example, a T4 DNA ligase (as available, for example, from New England Biolabs). Such an enzyme may catalyze the formation of a phosphodiester bond between juxtaposed 5′ phosphate and 3′ hydroxyl termini in duplex DNA or RNA. Suitable methods for the ligation of DNA and/or RNA molecules using a DNA ligase are known in the art and/or described in Ausubel et al (In: Current Protocols in Molecular Biology. Wiley Interscience, ISBN 047 150338, 1987) and Sambrook et al (In: Molecular Cloning: A Laboratory Manual, Cold Spring Harbor Laboratories, New York, Third Edition, 2001).

In one embodiment, a conjugate comprising a single stranded DNA or RNA and a nucleic acid of the invention (whether single or double stranded) may be produced using an RNA ligase, such as, for example T4 RNA ligase (as available from New England Biolabs). An RNA ligase may catalyze ligation of a 5′ phosphoryl-terminated nucleic acid donor to a 3′ hydroxyl-terminated nucleic acid acceptor through the formation of a 3′-5′ phosphodiester bond, with hydrolysis of ATP to AMP and PP.

In a further embodiment, a nucleic acid conjugate may be produced using a crosslinking reagent attached to one of the nucleic acids. Any crosslinking agent capable of covalently attaching two oligonucleotides may be used, for example, psoralen. Psoralen is a photoactivated crosslinking molecule with a rigid, flat structure that readily intercalates within a dsDNA or dsRNA double helix, preferably between an AT sequence. Both the furan and pyrone functional groups of the psoralen compound may be photolyzed with long wavelength UV light (365 nm) to form covalent bonds with particular nucleotide bases. The furan side is 4 times more reactive than the pyrone side and overwhelmingly favours reacting with T nucleotides. The furan and pyrone groups also both show reactivity with C and U nucleotides. Psoralen, psoralen derivatives and special phosphoramidites with 5′ psoralen linkers are commercially available (Glen Research). Using such a compound, a nucleic acid conjugate may be produced by contacting a psoralen linked nucleic acid with another nucleic acid for a time and under conditions sufficient for a covalent bond to form (e.g., as described supra). A suitable method for conjugating nucleic acids using psoralen is described, for example, in Kessler (1992) “Nonradioactive labeling methods for nucleic acids” in Kricka (ed.) Nonisotopic DNA Probe Techniques, Academic Press; and Geoghegan et al., Bioconjug Chem 3:138-146, 1992.

### Analogues and Derivatives of the Double-Stranded Oligonucleotides

The present invention encompasses use of any analogues and derivatives of the double-stranded oligonucleotides as described herein. For example, the oligonulceotides may be derivatized to include 5′- and/or 3′- insertions that do not adversely affect its ability to bind to a Ter binding polypeptide or a homologue, analogue or derivative thereof. Such insertions include the addition of mRNA and/or DNA that is to be presented or displayed.

### Analogues of Ribonucleotides and Deoxyribonucleotides

The present invention encompasses use of analogues of deoxyribonucleotides or ribonucleotides, for example, wherein the base is substituted for an analogous base having the same base-pairing attributes.

Analogues of a ribonucleotide or deoxyribonucleotide may comprise modifications to the phosphate and/or sugar and/or base. Modified phosphate groups may comprise non-hydrolyzable substituents, bis-nucleoside phosphates, or gamma-phosphate linkers, amongst others, or combinations thereof. Modified sugars may comprise one or more fluorescent substituents, nucleoside biphosphates, cyclic nucleotides, amino linkers, halogen or other heavy substituents (e.g., bromine, fluorine, chlorine, iodine, astatine), arabinose, amongst others, or combinations thereof. Modified bases may comprise one or more uncommon bases (e.g., inosine, xanthine, hypoxanthine, c-adenosine, ribavirin, dPTP, a 6-chloropurine substituent, a 6-mercaptopurine substituent), fluorescent substituents, thiol substituents (e.g., 6-thio-inosine-5′-triphosphate), amino linkers, halogen or other heavy substituents (e.g., bromine, fluorine, chlorine, iodine, astatine), amongst others, or combinations thereof. Caged nucleotide analogues incorporating one or more photolabile groups may also be employed. Such analogues are readily obtained from commercial sources e.g., Jena Bioscience GmbH, Loebstedter Str. 78, 07749 Jena, Germany.

Analogues may comprise alkylated (e.g., methylated), iodinated, brominated or biotinylated deoxyribonucleotides or ribonucleotide residues. Other analogues may also be used. For example, any one or more of A, C, G or T is substituted for a ribonucleotide or deoxyribonucleotide residue having the same or similar base-pairing ability and/or wherein T is substituted for an alkylated, biotinylated or halogenated ribonucleotide or deoxyribonucleotide having the same or similar base-pairing ability.

### Fluorescent Analogues

Fluorescent analogues may comprise one or more compact fluorophores that are particularly useful as they show only minimal effects on protein-nucleotide interactions due to their low molecular weight. When incorporated into the TT-Lock oligonucleotide of the present invention, the resultant oligonucleotide may be useful for stopped-flow and equilibrium analysis of nucleotide-protein interactions in kinetic studies, environmentally-sensitive fluorescence, fluorescence in-situ hybridization (FISH), ligand binding studies, energy transfer studies (FRET), fluorescence microscopy or X-ray crystallography, methods described, for example, by Hiratsuka Eur J Biochem 270:3479, 2003; Gille et al., NS Arch Pharmacol 368:210, 2003; Gille et al., NS Arch Pharmacol 369:141, 2004; Gromadski et al., Nat Struct Mol Biol 11:316, 2004). Exemplary substituents for such analogues may include N-methyl-anthraniloyl (i.e., mant);

4-(N-methyl-anthraniloyl)-amino (i.e., mant-amino); 4-(N-methyl-anthraniloyl)-amino)butyl (i.e., 4-(mant-amino)butyl); 6-(N-methyl-anthraniloyl)-amino)hexyl (i.e., 6-(mant-amino)hexyl); 2-(N-methyl-anthraniloyl)-amino)ethyl-carbamoyl (i.e., mant-EDA); 273′-(0-trinitrophenyl) (i.e., TNP); P³-(1-(2-nitrophenyl)-ethyl)-ester (i.e., NPE-caged substituent); methyl-7-guanosine (i.e., m⁷G) and the like.

Accordingly, exemplary fluorescent adenosine analogues suitable for such applications may include mant-ADP (2′/3′-O-(N-methyl-anthraniloyl)-adenosine-5′-diphosphate); mant-ATP (2′/3′-(N-methyl-anthraniloyl)-adenosine-5′-triphosphate); mant-N⁶-methyl-ATP (2′/3′-O-(N-Methyl-anthraniloyl)-N6-methyl-adenosine-5′-triphosphate); N⁶-[4-(mant-amino)]butyl-ATP (N⁶44-((N-methyl-anthraniloyl)-amino)]butyl-adenosine-54riphosphate); N⁶-[6-(mant-amino)]hexyl-ATP; 8-[4-(mant-amino)]butyl-ATP (MABA-ATP); 846-(mant-amino)Thexyl-ATP (MAHA-ATP); mant-EDA-ATP (273′-[(2-(N-methyl-anthraniloyl)-amino)ethyl-carbamoyl]-adenosine-5′-triphosphate); mant-dATP; 2′-mant-3′-dATP; mant-AppNHp (mant-AMPPNP); c-ATP (1,N⁶-etheno-ATP); E-AppNHp (1 ,N⁶-etheno-adenosine-5′-[(13,y)-imido]triphosphate or E⁻AMPPNP or 1 ,N⁶-etheno-AppNHp); TNP- ADP (2′/3′-(0-trinitrophenyI)-adenosine-5′-diphosphate); and TNP-ATP (273′-(0-trinitrophenyI)- adenosine-5′-triphosphate).

Exemplary fluorescent guanosine analogues may include mant-GDP; mant-dGDP; mant-GTP; mant-dGTP; NPE-caged-mant-dGTP; mant-GppNHp (mant-GMPPNP); mant-dGppNHp (mant-dGMPPNP); mant-GTPyS; TNP-GDP; TNP-GTP; TNP-GppNHp (TNP-GMPPNP); ant-GTP;

ant-m⁷GMP, ant-m⁷GDP; ant-m⁷GTP; and 2′-mant-3′-dGTP.

Exemplary fluorescent uridine or cytidine analogues may be 2′/3′-(0-trinitrophenyI)- uridine-5′-triphosphate (TNP-UTP) and 273¹-(0-trinitrophenyl)-cytidine-5′-triphosphate (TNP-CTP), respectively.

Exemplary fluorescent analogues of xanthine (X) or inosine (I) may include mant-XDP; mant-XTP; mant-XppNHp (mant-XMPPNP); and mant-ITPyS.

Non-Hydrolyzable Analogues

Exemplary non-hydrolyzable adenosine analogues may include ApCp (AMPCP); ApCpp (AMPCPP); AppCp (AMPPCP); AppNHp (AMPPNP); ATP(S; dATP(S; ATPyS; mant-AppNHp (mant-AMPPNP); NPE-caged-AppNHp (NPE-caged-AMPPNP); EDA-AppNHp (EDA-AMPPNP); biotin-EDA-AppNHp; (biotin-EDA-AMPPNP); 3-methylene-APS; E-AppNHp (E-AMPPNP or 1,N⁶- etheno-AppNHp); and AppNH2 (AMPPN). Exemplary non-hydrolyzable analogues of cytidine may include dCTP(S.

Exemplary non-hydrolyzable guanosine analogues may include GpCp (GMPCP); GpCpp (GMPCPP); NPE-caged-GpCpp (NPE-caged-GMPCPP); GppCp (GMPPCP); GppNHp (GMPPNP); GDP13S; GTP(S; dGTP(S; GTP(S; mant-GppNHp (mant-GMPPNP); mant-dGppNHp (mant-dGMPPNP); mant-GTPyS; 6-thio-GpCp (6-thio-GMPCP); 6-thio-GppCp (6-thio-GMPPCP);

6-thio-GppNHp (6-thio-GMPPNP); and TNP-GppNHp (TNP-GMPPNP).

Exemplary non-hydrolyzable analogues of thymidine may include dTTP(S. Exemplary non-hydrolyzable analogues of uridine may include UTP(S; UppNHp (UMPPNP); UTPyS; dUpNHp (dUMPNP); and dUpNHpp (dUMPNPP). Exemplary non-hydrolyzable analogues of xanthine or inosine may include XppCp;

(XMPPCP); XppNHp (XMPPNP); mant-XppNHp (mant-XMPPNP); NPE-caged-XppNHp (NPE- caged-XMPPNP); XTPyS; IppNHp (IMPPNP); ITPyS; and mant-ITPyS.

Halogenated analogues Exemplary halogenated analogues of adenosine may include 21-ADP, 2′Br-ADP; 8I-ADP;

8Br-ADP; 2′I-ATP; 2′Br-ATP; 81-ATP; 8Br-ATP; 21-AppNHp (21-AMPPNP); 2′Br-AppNHp (2′Br- AMPPNP); 81-AppNHp (81-AMPPNP); 8Br-AppNHp (8Br-AMPPNP); 8Br-cAMP; and 8Br-dATP.

Exemplary halogenated cytidine analogues may include 5I-dCTP; 5Br-CTP; 5Br-UMP; 5Br-dCMP; 5Br-dCDP; and 5Br-dCTP. Exemplary halogenated guanosine analogues may include 8I-GDP; 8Br-GDP; 81-GTP;

8Br-GTP; 8I-GppNHp (8I-GMPPNP); and 8Br-GppNHp (8Br-GMPPNP).

Exemplary halogenated uridine analogues may include 5I-dUMP; 5I-UTP; 51-dUTP (5′IdU); 5Br-UTP; 5Br-dUDP (5′BrdU); 5Br-dUTP; and 5F-UTP.

Exemplary halogenated thymidine analogues may include 5I-dUMP; 5I-UTP; 5I-dUTP (5¹1dU); 5Br-UTP; 5Br-dUDP (5′BrdU); 5Br-dUTP; and 5F-UTP.

Amine-labeled analogues

Exemplary amine-labeled analogues of adenosine may include N⁶-(4-amino)butyl-ATP; N⁶-(6-amino)hexyl-ATP; 8-[(4-amino)butyl]-amino-ATP; 8-[(6-amino)hexyl]-amino-ATP; EDA-ADP; EDA-ATP; EDA-AppNHp (EDA-AMPPNP); y-aminophenyl-ATP; y-aminohexyl-ATP; y-aminooctyl- ATP; y-aminoethyl-AppNHp (y-aminoethyl-AMPPNP); 8-[(6-amino)hexyl]-amino-adenosine-2′,5′- bisphosphate; and 8-[(6-amino)hexyl]-amino-adenosine-3′,5′-bisphosphate.

Exemplary amine-labeled guanosine analogues may include y-aminohexyl-GTP; y- aminooctyl-GTP; EDA-GTP; y-aminohexyl-m⁷GTP; EDA-m⁷GTP; and EDA-m⁷GDP.

Thiol analogues

Exemplary thiol guanosine analogues may include 6-thio-GTP; 6-thio-GpCp (6-thio- GMPCP); 6-thio-GppCp (6-thio-GMPPCP); 6-thio-GppNHp (6-thio-GMPPNP); 6-methylthio-GMP;

6-methylthio-GDP; 6-methylthio-GTP; 6-thio-GMP; and 6-thio-GDP.

Exemplary thiol inosine analogues may include 6-methylthio-IMP; 6-methylthio-IDP; 6- methylthio-ITP; and 6-mercaptopurine-riboside-5¹-triphosphate (6-thio-inosine-5′-triphosphate).

Biotinylated analogues

Exemplary biotinylated nucleotide analogues may include biotin-EDA-AppNHp; (biotin- EDA-AMPPNP); biotin-EDA-ATP; and biotin-EDA-AppNHp (biotin-EDA-AMPPNP). Exemplary biotinylated uridine analogues may include biotin-XX-UTP.

2′-deoxyuridine analogues

Exemplary 2′-deoxyuridine analogues may include dUDP; 5Br-dUDP; dUTP; 5Br-dUTP; dUpNHp (dUMPNP); dUpNHpp (dUMPNPP); 5l-dUTP; aminoallyl-dUpCp (aminoallyl-dUMPCP); and aminoallyl-dUpCpp (aminoallyl-dUMPCPP).

Other suitable analogues

Other suitable adenosine analogues may include 13-methylene-APS; biotin-EDA-ATP; biotin-EDA-AppNHp (biotin-EDA-AMPPNP); 8Br-cAMP; adenosine-3′,5′-bisphosphate; adenosine- 2′,5′-bisphosphate; 2′-0-methyl-adenosine-3′,5′-bisphosphate (2′OMe-pAp); N⁶-methyl-ATP; AP4 (adenosine-5′-tetraphosphate); ara-ATP; and 3′-dATP.

Other suitable cytidine analogues may include 5-methyl-dCTP; 5-aza-dCTP; 3TCMP; and 3TCTP.

Other suitable guanosine analogues may include cGMP; guanosine-3′,5′-bisphosphate (pGp); guanosine-2′,5′-bisphosphate; 8-oxo-GTP; 8-oxo-dGTP; m⁷GTP; and 2′-0-methyl-GTP (2′0Me-GTP). Other suitable thymidine analogues may include AzTMP; AzTTP; d4TMP; daTTP.

Ter binding polypeptides and analogues and derivatives thereof

The amino acid sequence of an E. coli Ter binding polypeptide is shown in SEQ ID NO: 5. The percentage identity to SEQ ID NO: 5 may be at least about 85%, more preferably at least about 90%, even more preferably at least about 95% and still more preferably at least about 99%.

For example, the Escherichia coli Ter binding protein is known in the art to be a monomeric 36-kDa protein that forms a simple 1:1 complex with a Ter site, as reviewed for example, by Hill, In: Escherichia coli and Salmonella: Cellular and Molecular Biology (Neidhardt

FC, ed) Vol 2, pp 1602-1614, Am. Soc Microbiol, Washington DC, USA and in Neylon et al., Microbiol Mol Biol Rev 69:501-526, 2005.

Cross-linking It is contemplated in the present invention that methods described herein may contain steps which involve irreversible and regionally-specific crosslinking of a Ter binding protein or mutant thereof with a Ter analogue, derivative or fragment thereof, for use as a signal generation system and to link this generation system to an anti-target molecule (e.g. a protein).

The present invention encompasses fusion proteins or conjugate of a Ter binding is polypeptide having Ter binding activity, for example, linked to a protein of interest. Such a fusion protein may be useful, for example, for displaying, detecting, identifying, amplifying and/or quantifying a protein of interest such as a protein (e.g. a biomarker). Thus, the fusion protein may be contacted to a solid surface coated with a TT-Lock nucleic acid of the invention for a time and under conditions for binding to occur, thereby displaying the protein of interest on the solid surface for, for example, use in an immunoassay.

The peptide, polypeptide or protein of interest may be fused to either end of the Ter binding protein or analogue, homologue or fragment with Ter binding activity or even conjugated to an internally region of the Ter binding polypeptide. The peptide, polypeptide or protein of interest and the Ter binding polypeptide may be capable of folding correctly and maintaining their distinct activities. Methods for fusing two or more proteins are known in the art and described, for example, in Scopes (In: Protein Purification: Principles and Practice, Third Edition, Springer Verlag, 1994).

For example, two proteins may be linked by virtue of formation of a disulphide bond between a cysteine residue in each of the proteins. Should a protein comprise multiple cysteine residues, any of these cysteine residues may be replaced when they occur in parts of a polypeptide where their participation in a cross-linking reaction would likely interfere with biological activity. When a cysteine residue is replaced, it may be desirable to minimize resulting changes in polypeptide folding. Changes in polypeptide folding may be minimized when the replacement is chemically and sterically similar to cysteine, such as, for example, serine. Alternatively, or in addition, a cysteine residue may be introduced into a polypeptide for cross-linking purposes. The cysteine residue may be introduced at or near the amino- or carboxy-terminus of the peptide or polypeptide. Methods for the production of a polypeptide comprising a suitable cysteine residue, for example, a recombinant protein, will be apparent to the skilled artisan. Following production of the polypeptides comprising suitable cysteine residues, cysteine residues may be oxidised using, for example, Cu(II)-(1,10-phenanthroline)₃ (CuPhe). The proteins may then be crosslinked using, for example, a dimaleimide (e.g., N,N″-o-phenylenedimaleimide (o- PDM), N,N″-p-phenylenedimaleimide (p-PDM) or bismaleimidohexane (BMH)). Following quenching of the reaction (e.g., with DTT) cross-linked proteins may be isolated. Alternatively, io photocross-linking of cysteine residues may be performed, for example, as described in Giron- Morzon et al., J Biol Chem 279:49338-49345, 2004.

In another embodiment, coupling of the two polypeptide constituents (or a polypeptide and another compound, for example, a small molecule) may be achieved using a coupling or conjugating agent, such as for example, a chemical cross-linking agent. Methods for the use of a chemical cross-linking reagent are known in that art and reviewed, for example, in Means et al.,

Bioconjugate Chemistry 1:2-12, 1990.

There are several intermolecular crosslinking reagents useful for the performance of the instant invention (see, for example, Means, G. E. and Feeney, R. E., Chemical Modification of Proteins, Holden-Day, 1974, pp. 39-43). Among these reagents are, for example, J-succinimidyl 3- (2-pyridyldithio)propionate (SPDP) or N,N′-(1,3-phenylene)bismaleimide (both of which are highly specific for sulfhydryl groups and form irreversible linkages); N,N′-ethylene-bis-(iodoacetamide) or other such reagent having 6 to 11 carbon methylene bridges (which are relatively specific for sulfhydryl groups); and 1,5-difluoro-2,4-dinitrobenzene (which forms irreversible linkages with amino and tyrosine groups). Other crosslinking reagents useful for this purpose may include: p,p′- difluoro-m,m′-dinitrodiphenylsulfone (which forms irreversible cross-linkages with amino and phenolic groups); dimethyl adipimidate (which is specific for amino groups); phenol-1,4- disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and bisdiazobenzidine (which reacts primarily with tyrosine and histidine).

In this regard, a cross-linking reagent may be homobifunctional that is, having two functional groups that undergo the same reaction. Homobifunctional crosslinking reagent may be bismaleimidohexane (BMH). BMH contains two maleimide functional groups, which may react specifically with sulfhydryl-containing compounds under mild conditions (pH 6.5-7.7). The two maleimide groups are connected by a hydrocarbon chain. Accordingly, BMH may be useful for irreversible attachment of a polypeptide to another molecule that contains one or more cysteine residues.

Alternatively, a crosslinking reagent may be heterobifunctional. A heterobifunctional crosslinking agent may have two different functional groups, for example, an amine-reactive group and a thiol-reactive group that will cross-link two molecules having free amines and thiols, respectively. Such a heterobifunctional crosslinker may be useful for specific coupling methods for conjugating two chemical entities, thereby reducing the occurrences of unwanted side reactions such as homo-protein polymers. A variety of heterobifunctional crosslinkers are known in the art. Examples of heterobifunctional crosslinking agents may include succinimidyl 4-(N- maleim idomethyl)-cyclohexane-1 -carboxylate (SMCC), N-succinimidyl(4-iodoacetyl) aminobenzoate (STAB), 1-ethyl-3-(3-dimethylaminopropyl) carbodiimide hydrochloride (EDC); 4- succinimidyloxycarbonyl-a-methyl-a-(2-pyridyldithio)-toluene (SMPT), N-succinimidyl 3-(2- pyridyldithio)propionate (SPDP), succinimidyl 6-[3-(2-pyridyldithio)propionate] hexanoate (LC- SPDP), m-maleimidobenzoyl-N-hydroxysuccinimide ester (MBS), and succinimide 4-(p- maleimidophenyl)butyrate (SMPB), an extended chain analog of MBS. The succinimidyl group of these crosslinkers may react with a primary amine, and the thiol-reactive maleimide may form a covalent bond with the thiol of a cysteine residue.

In addition, photoreactive crosslinkers, such as, for example bis-[®-(4- 2o azidosalicylamido)ethyl]disulfide (BASED) and N-succinimidyl-6(4′-azido-2′-nitrophenyl- amino)hexanoate (SANPAH) may be useful for producing a protein conjugate.

As will be apparent from the foregoing, the present invention contemplates production of a protein conjugate by performing a process comprising contacting a Ter binding protein with Ter binding activity and a peptide, polypeptide or protein of interest with a compound capable of forming a bond between two proteins for a time and under conditions sufficient to form a bond thereby producing a conjugated protein.

The reagents described above are additionally useful for linking a protein to a non- proteinaceous compound, for example, a small molecule. In particular, the chemical cross-linking reagents described herein and known in the art may be useful for linking a Ter binding polypeptide with Ter binding activity to a compound of interest.

Preparation and/or use of fusion proteins

The present invention further encompasses the preparation and/or use of fusion proteins of a Ter binding protein having Ter binding activity, for example, linked to a protein of interest.

Such a fusion protein may be useful, for example, for displaying a protein of interest. For example, the conjugate protein may be contacted to a solid surface coated with a 7-Lock nucleic acid of the invention for a time and under conditions for binding to occur, thereby displaying the protein of interest on the solid surface for, for example, use in an immunoassay such as a competitive immunoassay or a noncompetitive immunoassay.

It is contemplated herein that a competitive immunoassay may involve the presence of an antigen in the unknown sample which competes with labeled antigen (for example a TUS fusion) to bind with antibodies. The amount of labeled antigen bound to the antibody site is then io measured. In this method, the response will be inversely proportional to the concentration of antigen in the unknown. This is because the greater the response, the less antigen in the unknown was available to compete with the labeled antigen.

It is contemplated herein that noncompetitive immunoassays which can be also referred to as the “sandwich assay,” may involve an antigen in the unknown sample which is bound to an antibody site, that is labeled antibody is bound to the antigen. The amount of labeled antibody on the site is then measured. Unlike the competitive method, the results of the noncompetitive method will be directly proportional to the concentration of the antigen. This is because labeled antibody will not bind if the antigen is not present in the unknown sample.

The peptide, polypeptide or protein of interest may be fused to either end of the Ter binding protein with Ter binding activity or fused to an internal region of the Ter binding protein.

The peptide, polypeptide or protein of interest and the Ter binding protein may be capable of folding correctly and maintaining their distinct activities. Methods for conjugating two or more proteins are known in the art and described, for example, in Scopes (In: Protein Purification: Principles and Practice, Third Edition, Springer Verlag, 1994). The present invention additionally contemplates the production of a fusion protein that comprises a Ter binding protein and a peptide, polypeptide or protein of interest. Methods for the production of a fusion protein are known in the art and described, for example, in Ausubel et al (In: Current Protocols in Molecular Biology. Wiley Interscience, ISBN 047 150338, 1987) and Sambrook et al (In: Molecular Cloning: A Laboratory Manual, Cold Spring Harbor Laboratories, New York, Third Edition 2001).

The present invention further contemplates the production of a fusion protein that comprises a Ter binding protein and a peptide, polypeptide or protein of interest, together with a molecular tag, wherein said tag is suitable for immobilization of said fusion protein.

The tag may be selected from the group comprising hexa-histidine (His6), biotin ligase substrate sequences, FLAG, maltose binding protein or glutathione S transferase (GST). The tag may be His6 or biotin ligase substrate sequences. Other tags comprising a Ter binding polypeptide fused to a peptide, polypeptide or protein of interest are also contemplated by the present invention. General methods for producing a recombinant fusion protein involve the production of nucleic acid that encodes said fusion protein. In this regard, the present invention provides a nucleic acid encoding a fusion protein comprising a Ter binding protein with Ter binding activity and a peptide, polypeptide or protein of interest. The fusion protein may be an in frame fusion.

The nucleic acid encoding the constituent components of the fusion protein may be isolated using a known method, such as, for example, amplification (e.g., using PCR or splice overlap extension) or isolated from nucleic acid from an organism using one or more restriction enzymes or isolated from a library of nucleic acids or synthesized using a method known in the art and/or described herein. Methods for such isolation will be apparent to the ordinary skilled artisan.

For example, nucleic acid (e.g., genomic DNA or RNA that is then reverse transcribed to form cDNA) from a cell or organism comprising a protein of interest may be isolated using a method known in the art and cloned into a suitable vector. The vector may then be introduced into a suitable organism, such as, for example, a bacterial cell. Using a nucleic acid probe from the gene encoding the protein of interest, a cell comprising the nucleic acid of interest may be isolated using methods known in the art and described, for example, in in Ausubel et al (In: Current Protocols in Molecular Biology. Wiley Interscience, ISBN 047 150338, 1987), Sambrook et al (In:

Molecular Cloning: A Laboratory Manual, Cold Spring Harbor Laboratories, New York, Third Edition 2001).

Alternatively, nucleic acid encoding a protein of interest may be isolated using polymerase chain reaction (PCR). Methods of PCR are known in the art and described, for example, in Dieffenbach (ed) and Dveksler (ed) (In: PCR Primer: A Laboratory Manual, Cold Spring Harbour

Laboratories, NY, 1995). Generally, for PCR two non-complementary nucleic acid primer molecules comprising at least about 20 nucleotides in length, and more preferably at least 25 nucleotides in length may be hybridized to different strands of a nucleic acid template molecule, and specific nucleic acid molecule copies of the template may be amplified enzymatically. The primers may hybridize to nucleic acid adjacent to a gene or coding region encoding the protein of interest, thereby facilitating amplification of the nucleic acid that encodes the subunit. Following amplification, the amplified nucleic acid may be isolated using methods known in the art.

Other methods for the production of an oligonucleotide of the invention will be apparent to the skilled artisan and are encompassed by the present invention. Following isolation of each of the components of the fusion protein, a fusion protein encoding nucleic acid may be produced, for example, by ligating the two coding regions together in frame such that a single protein is produced, e.g., using a DNA ligase. Alternatively, an amplification reaction may be performed using one or more primers that are capable of hybridizing to both components and thereby produce a single nucleic acid molecule.

The nucleic acid may additionally include regions that encode, for example, a linker or spacer region, a detectable marker and/or a further fusion protein. For example, a nucleic acid encoding a linker or spacer region may be included between the Ter binding protein with Ter binding activity and the peptide, polypeptide or protein to facilitate correct folding of each of the constituent components of the fusion protein. The linker may have a high freedom degree for linking of two proteins, for example a linker comprising glycine and/or serine residues. Suitable linkers are described, for example, in Robinson and Sauer, Proc Natl Acad Sci USA 95:5929- 5934, 1998, or Crasto and Fang, Protein Engineering 13:309-312, 2000.

Following isolation of the nucleic acid encoding the fusion protein, an expression construct that comprises nucleic acid encoding the fusion protein of the invention may be produced. As will be apparent from the foregoing, an expression construct useful for the production of a fusion protein of the invention may comprise a promoter. The nucleic acid comprising the promoter sequence may be isolated using a technique known in the art, such as for example PCR or restriction digestion. Alternatively, the nucleic acid comprising the promoter sequence may be synthetic, for example, an oligonucleotide.

Placing a nucleic acid molecule under the regulatory control of a promoter sequence may involve positioning said molecule such that expression is controlled by the promoter sequence. Promoters are generally positioned 5′ (upstream) to the coding sequence that they control. To construct heterologous promoter/structural gene combinations, the promoter may be positioned at a distance from the gene transcription start site that is approximately the same as the distance between that promoter and the gene it controls in its natural setting, that is, the gene from which the promoter is derived. As is known in the art, some variation in this distance may be accommodated without loss of promoter function. Similarly, the preferred positioning of a regulatory sequence element with respect to a heterologous gene to be placed under its control may be defined by the positioning of the element in its natural setting, that is, the gene from which it is derived. As is known in the art, some variation in this distance can also occur.

Should it be preferred that the fusion protein be expressed in vitro, a suitable promoter may include, but is not limited to, the T3 or T7 bacteriophage promoters (Hanes and PlOckthun, Proc Natl Acad Sci USA, 94:4937-4942, 1997). Typical expression vectors for in vitro expression or cell-free expression have been described and include, but are not limited to the TNT T7 and TNT T3 systems (Promega), the pEXP1-DEST and pEXP2-DEST vectors (Invitrogen).

Typical promoters suitable for expression in bacterial cells include, but are not limited to, the lacZ promoter, the Ipp promoter, temperature-sensitive XpL or XpR promoters, T7 promoter,

T3 promoter, SP6 promoter or semi-artificial promoters such as the IPTG-inducible tac promoter or lacUV5 promoter. A number of other gene construct systems for expressing the nucleic acid fragment of the invention in bacterial cells are known in the art and are described for example, in Ausubel et al. (In: Current Protocols in Molecular Biology, Wiley Interscience, ISBN 047 150338, 1987), US Patent No. 5,763,239 (Diversa Corporation) and Sambrook et al. (In: Molecular Cloning:

A Laboratory Manual, Cold Spring Harbor Laboratories, New York, Third Edition, 2001).

Numerous expression vectors for expression of recombinant polypeptides in bacterial cells and efficient ribosome binding sites have been described, and include, for example, pKC30 (Shimatake and Rosenberg, Nature 292:128, 1981); pKK173-3 (Amann and Brosius, Gene 40:183, 1985), pET-3 (Studier and Moffat, J Mol Biol 189:113, 1986); the pCR vector suite

(Invitrogen), pGEM-T Easy vectors (Promega), the pBAD/TOPO (Invitrogen), the pFLEX series of expression vectors (Pfizer Inc., CT,USA), the pQE series of expression vectors (QIAGEN, CA, USA), or the pL series of expression vectors (Invitrogen), amongst others.

Typical promoters suitable for expression in a mammalian cell, mammalian tissue or intact mammal include, for example a promoter selected from the group consisting of, a retroviral LTR element, a SV40 early promoter, a SV40 late promoter, a cytomegalovirus (CMV) promoter, a CMV IE (cytomegalovirus immediate early) promoter, an EF1 a promoter (from human elongation factor 1 a), an EM7 promoter, a T7 promoter (from bacteriophage T7), a lambda promoter (from Lambda bacteriophage) or an UbC promoter (from human ubiquitin C). Expression vectors that contain suitable promoter sequences for expression in mammalian cells or mammals include, but are not limited to, the pcDNA vector suite supplied by Invitrogen, the pCI vector suite (Promega), the pCMV vector suite (Clontech), the pM vector (Clontech), the pSI vector (Promega) or the VP16 vector (Clontech).

As will be apparent from the foregoing, the present invention provides a method for producing an expression construct encoding a fusion protein of the invention comprising placing a nucleic acid encoding the fusion protein in operable connection with a promoter.

Furthermore, the present invention provides a vector comprising a nucleic acid encoding a fusion protein comprising a Ter binding polypeptide or an analogue, homologue or fragment thereof and a peptide, polypeptide or protein of interest.

Following production of a suitable expression construct, a recombinant fusion protein may be produced. This may involve introducing the expression construct into a cell for expression of the recombinant protein. Methods for introducing an expression construct into a cell for expression s are known to those skilled in the art and are described for example, in Ausubel et al. (In: Current

Protocols in Molecular Biology. Wiley Interscience, ISBN 047 150338, 1987) and Sambrook et al. (In: Molecular Cloning: A Laboratory Manual, Cold Spring Harbor Laboratories, New York, Third Edition, 2001). The method chosen to introduce the gene construct depends upon the cell type in which the gene construct is to be expressed. Means for introducing recombinant DNA into cells include, but are not limited to electroporation, chemical transformation into cells previously treated to allow for said transformation, PEG mediated transformation, microinjection, transfection mediated by DEAE-dextran, transfection mediated by calcium phosphate, transfection mediated by liposomes such as by using Lipofectamine (Invitrogen) and/or cellfectin (Invitrogen), transduction by Adenoviuses, Herpesviruses, Togaviruses or Retroviruses and microparticle bombardment is such as by using DNA-coated tungsten or gold particles (Agacetus Inc., WI, USA).

Following transformation or transfection, cells may be incubated for a time and under conditions sufficient for expression of the fusion protein. If a purified fusion protein is desired, the protein may then be isolated by a method known in the art, such as, for example, by affinity purification. Methods for the isolation of a protein are known in the art and/or described in Scopes (In: Protein Purification: Principles and Practice, Third Edition, Springer Verlag, 1994).

In an alternative embodiment, the fusion protein may be produced in vitro, using an in vitro expression system as described above. Such a system may be used to translate a previously produced RNA molecule, for example, using a rabbit reticulocyte lysate (available from Promega Corporation) or to transcribe and/or translate a nucleic acid construct (e.g., a DNA construct), for example, using an E. coli extract (also available from Promega Corporation). Various kits for in vitro transcription/translation are commercially available. Following in vitro production, the fusion protein may be isolated or purified using, for example, affinity purification.

Surface plasmon resonance chips The inventors have developed and described herein a real-time exonuclease assay based on use of a sensor, in particular a chip. It is contemplated herein that the chip may be a surface plasmon resonance (SPR; Biacore) sensor that can bind oligonucleotides in a reversible fashion. Accordingly, the present invention provides for a chip, wherein the chip comprises the double- stranded oligonucleotides or the conjugates described herein.

Display formats

In one embodiment, the double-stranded oligonucleotides of the present invention may be used for in vitro display, such as ribosome display, ribosome inactivation, covalent display or mRNA display.

The present invention accordingly provides methods and processes for identifying, detecting, amplifying and/or quantifying a target molecule such as a polypeptide, nucleic acid, antibody or small molecule on a surface, said method comprising linking a fusion protein to an oligonucleotide, wherein the fusion protein has an affinity to a target molecule, said method further comprising contacting target molecule to an immobilised molecule on the surface wherein the immobilised molecule can bind to the target molecule under conditions sufficient to identify, detect, amplify and/or quantify a target molecule on the surface.

In addition, the present invention accordingly provides methods for presenting or displaying a molecule such as a polypeptide, nucleic acid, antibody or small molecule on a surface, said method comprising contacting a conjugate comprising the oligonucleotide as described herein covalently bound to the molecule with a Ter binding polypeptide bound to the surface for a time and under conditions sufficient to form a DNA/protein complex wherein the molecule is displayed on the surface.

Optionally, the method further comprises cross-linking the double-stranded nucleic acid moiety of the conjugate to the Ter binding polypeptide or a homologue, analogue or derivative thereof, for example, using formaldehyde.

Optionally, the method further comprises cross-linking the double-stranded nucleic acid moiety of the conjugate to the Ter binding polypeptide or a homologue, analogue or derivative thereof, for example, using photochemical crosslinking as described herein. These embodiments may be particularly suitable for presenting or displaying nucleic acid, in which case the conjugate comprises the double-stranded oligonucleotides bound to DNA or RNA. However, it is to be understood that this embodiment of the invention is also useful for presenting or displaying any other molecule capable of being conjugated to nucleic acid, particularly to single-stranded or double-stranded DNA. For example, the oligonucleotides of the present invention may be conjugated to a protein for use in a forward or reverse hybrid assay (e.g., to identify a ligand of a protein or to identify a receptor agonist or antagonist) or immunoassay (e.g., ELISA), or to an antibody for use such as for use in epitope mapping or immunoassay, or to a small molecule for use in screening applications (e.g., to screen for an agonist or antagonist of a receptor protein). Other applications are not to be excluded. The surface may be any surface suitable for nucleic acid hybridization (RNA/DNA,

RNA/RNA or DNA/DNA hybridization) or for analysing the interaction of a nucleic acid, protein, antibody or small molecule with nucleic acid. As will be known to the skilled artisan, this may include the surface of a microwell or a glass, nylon or composite material suitable for producing a microarray, a polymeric pin, or chromatographic material e.g., agarose, Sepharose, cellulose, polyacrylamide, etc.

The surface may be prepared or provided in a ready-to-use format and the present invention encompasses the preparation of the surface for use. Accordingly, in one embodiment, the method further comprises the first step of contacting the surface with the Ter binding io polypeptide, homologue, analogue or derivative for a time and under conditions sufficient for said polypeptide to bind to said surface. The binding may be covalent or non-covalent, for example, electrostatic or van der Waals interaction.

Subject to the proviso that the double-stranded oligonucleotide has not been cross-linked to a Ter binding polypeptide, the surface, once prepared, is readily reusable. Accordingly, in another preferred embodiment, the method further comprises disrupting the DNA/protein complex and contacting a conjugate comprising a double-stranded oligonucleotide as described herein covalently bound to a molecule (e.g., a second molecule different to the first molecule) with the Ter binding polypeptide having TerB binding activity for a time and under conditions sufficient to form a DNA/protein complex wherein the molecule is displayed on the surface by virtue of said interaction.

The invention also encompasses such display formats in the reverse or opposite format wherein the oligonucleotides of the invention are bound to a surface and a conjugate comprising a Ter binding polypeptide is bound reversibly or irreversibly thereto. Such a reverse format may be suitable for presenting or displaying any polypeptide or peptide that can be produced as a fusion polypeptide with Tus or chemically added thereto, for example, in preparation for a forward or reverse hybrid assay (for example, to identify a ligand of a protein or to identify a receptor agonist or antagonist) or immunoassay (e.g., ELISA). However, it is to be understood that any other molecule capable of being conjugated to protein may be displayed in accordance with this embodiment. For example, a Ter binding protein may be conjugated to a nucleic acid for use in a hybridization assay. Alternatively, a Ter binding protein may be conjugated to an antibody for use in epitope mapping or an immunoassay, or to a small molecule for use in screening applications (for example, to screen for an agonist or antagonist of a receptor protein). Other applications are not to be excluded.

Accordingly, a further embodiment of the present invention provides a method for presenting or displaying a molecule such as a polypeptide, nucleic acid, antibody or small molecule on a surface, said method comprising contacting a conjugate comprising a Ter binding polypeptide having TerB binding activity covalently bound to the molecule to a double-stranded oligonucleotide as described herein bound to the surface for a time and under conditions sufficient to form a DNA/protein complex, wherein the molecule is displayed on the surface by virtue of said interaction.

The surface may be the surface of a microwell or a glass, nylon or composite material suitable for producing a microarray, a polymeric pin, or chromatographic material, for example, agarose, Sepharose, cellulose or polyacrylamide. The oligonucleotide may be bound to the io surface by any means, e.g., by cross-linking or other covalent attachment or by electrostatic interaction with the surface, the only requirement being that it is capable of binding to a Ter binding polypeptide when bound to the surface.

Optionally, the method further comprises cross-linking the double-stranded oligonucleotide moiety of the conjugate to the Ter binding polypeptide, for example, by using formaldehyde or by a photochemical reaction.

The surface may be prepared or provided in a ready-to-use format and the present invention therefore encompasses the preparation of the surface for use. In one preferred embodiment, the method further comprises the first step of contacting the surface with the double- stranded oligonucleotides as described herein for a time and under conditions sufficient for said oligonucleotide to bind to said surface.

Subject to the proviso that the Ter binding polypeptide conjugate has not been cross- linked to the double-stranded oligonucleotide, the surface may be reused. Accordingly, in a preferred embodiment, the method further comprises disrupting the DNA/protein complex and contacting a conjugate comprising a Ter binding polypeptide having TerB binding activity covalently bound to a molecule (for example, a second molecule different to the first molecule) with the oligonucleotide for a time and under conditions sufficient to form a DNA/protein complex, wherein the molecule is displayed on the surface by virtue of said interaction.

In other embodiments, the double-stranded oligonucleotides of the present invention may be used in a method of displaying mRNA or a polypeptide molecule or a conjugate comprising mRNA and a polypeptide encoded by it, wherein the mRNA or polypeptide molecule is displayed as part of a conjugate with the nucleic acid, or alternatively, as a capture reagent to assist in recovery of an mRNA or a polypeptide displayed as part of a conjugate with a Ter binding protein. The mRNA or polypeptide may be displayed on the surface of a ribosome,

For example, the present invention provides a method of presenting or displaying a molecule comprising incubating a conjugate comprising a double-stranded oligonucleotide as described herein covalently bound to mRNA for a time and under conditions sufficient for partial or complete translation of the mRNA to occur, thereby producing a complex comprising the conjugate, a nascent polypeptide encoded by the mRNA and optionally a ribosome. It is within the scope of the present invention for the conjugate to be covalently linked to puromycin for terminating translation. Alternatively, or in addition, the conjugate may be linked to a psoralen moiety to facilitate cross-linking of the mRNA to the nascent polypeptide.

Translation may be inactivated or stalled by contacting the incubating conjugate with a Ter binding polypeptide for a time and under conditions sufficient for the double-stranded i 0 oligonucleotide to bind to the Ter binding polypeptide, thereby stalling translation. Optionally, the double-stranded oligonucleotide moiety of the conjugate in the stalled translation mixture may be cross-linked to Ter binding polypeptide, for example, using formaldehyde, to stabilize the complex.

Alternatively, or in addition, the complex between the mRNA conjugate, a nascent polypeptide encoded by the mRNA and optionally a ribosome may be stabilized by addition of a reagent such as, for example, magnesium acetate or chloramphenicol.

Kits

The present invention also provides kits for producing the double-stranded nucleic acid molecule as described above, and for presenting or displaying a molecule, wherein the kits facilitate the employment of the methods and processes of the invention. Typically, kits for carrying out a method of the invention contain all the necessary reagents to carry out the method. Typically, the kits of the invention will comprise one or more containers, containing for example, wash reagents, and/or other reagents capable of releasing a bound component from a polypeptide or fragment thereof. In the context of the present invention, a compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept a test sample, a container which contains the polymers used in the assay and containers which contain wash reagents (such as phosphate buffered saline, Tris-buffers, and like).

Typically, a kit of the present invention will also include instructions for using the kit components to conduct the appropriate methods.

Methods and kits of the present invention find application in any circumstance in which it is desirable to purify any component from any mixture.

The present invention provides kits comprising a first strand oligonucleotide or an analogue or derivative thereof, and a second strand oligonucleotide or an analogue or derivative thereof, wherein said first strand oligonucleotide or analogue or derivative and said second strand oligonucleotide or analogue or derivative are in a form suitable for their annealing to produce the double-stranded nucleic acid molecule as described above.

The oligonucleotide or an analogue or derivative thereof may be provided in solution or as a solid e.g., a precipitate, or bound directly or indirectly to a solid matrix (e.g., a microwell, glass, nylon or composite material suitable for microassay, including a BlAcore chip, protein display chip, glass bead, microdot or quantum dot), a proteinaceous molecule, nucleic acid or small molecule. For example, the double-stranded oligonucleotide of the present invention can be bound covalently or cross-linked to a nucleic acid (e.g., mRNA), polypeptide (e.g., puromycin) or small molecule (e.g., psoralen, pyrido[3,4-c]psoralen or 7-methylpyrido[3,4-c]-psoralen). Alternatively, or in addition, the double-stranded oligonucleotide of the present invention can be bound non- covalently to a Ter binding protein or a homologue, analogue or derivative thereof.

The present invention further provides kits for detecting a target molecule from a sample of a subject in a monoplex or multiplex format comprising a first strand oligonucleotide or an analogue or derivative thereof, and a second strand oligonucleotide or an analogue or derivative thereof, wherein said first strand oligonucleotide or analogue or derivative and said second strand oligonucleotide or analogue or derivative are in a form suitable for their annealing to produce a double-stranded oligonucleotide wherein:

(a) said first strand comprises the sequence:

| | |
|---|---|
| 5′-N_{C} R N_{D} G T T G T A A C N_{D} A-3′ | (SEQ ID NO: 1) |

or an analogue or derivative of said sequence; and (b) said second strand comprises the sequence:

| | |
|---|---|
| 5′-T N_{D} G T T A C A A C N_{D} T N_{C} C-3′ | (SEQ ID NO: 2) |

or an analogue or derivative of said sequence wherein R is a purine, Nc and ND are each a DNA or RNA residue or analogue thereof, No residues in said first strand and said second strand are sufficiently complementary to permit said No residues to be annealed in the double-stranded oligonucleotide, and the sequence 5′- GTTGTAAC-3′ (SEQ ID NO: 3) of said first strand is annealed to the complementary sequence 5′- GTTACAAC-3′ (SEQ ID NO: 4) of said second strand.

The present invention further provides kits for detecting a target molecule from a sample obtained from a subject in a monoplex or multiplex format, wherein said kit comprises a first strand oligonucleotide or an analogue or derivative thereof, and a second strand oligonucleotide or an analogue or derivative thereof, wherein said first strand oligonucleotide or analogue or derivative and said second strand oligonucleotide or analogue or derivative are in a form suitable for their annealing to produce a double-stranded oligonucleotide wherein:

(a) said first strand comprises the sequence:

| | |
|---|---|
| 5′-N_{C} R N_{D} G T T G T A A C N_{D} A-3′ | (SEQ ID NO: 1) |

or an analogue or derivative of said sequence; and (b) said second strand comprises the sequence:

| | |
|---|---|
| 5′-T N_{D} G T T A C A A C N_{D} T N_{C} C-3′ | (SEQ ID NO: 2) |

or an analogue or derivative of said sequence wherein R is a purine, Nc and ND are each a DNA or RNA residue or analogue thereof, N_{D} residues in said first strand and said second strand are sufficiently complementary to permit said ND residues to be annealed in the double-stranded oligonucleotide, and the sequence 5′- GTTGTAAC-3′ (SEQ ID NO: 3) of said first strand is annealed to the complementary sequence 5′- GTTACAAC-3′ (SEQ ID NO: 4) of said second strand in a form suitable for conjugating to a second molecule, wherein said second molecule comprises a nucleic acid, polypeptide or small molecule. The present invention further provides kits for presenting or displaying a first molecule, wherein said first molecule comprises a double-stranded nucleic acid molecule as described above, in a form suitable for conjugating to:

(a) a second molecule, wherein said second molecule comprises a nucleic acid, polypeptide or small molecule; and (b) an integer selected from the group consisting of:

(i) a Ter binding polypeptide or a homologue, analogue or derivative thereof in a form suitable for conjugating to another molecule, wherein said double-stranded nucleic acid molecule and said Ter binding polypeptide interact in use to present or display another molecule conjugated to said double-stranded nucleic acid molecule or said polypeptide; and (ii) mRNA encoding a Ter binding polypeptide or a homologue, analogue or derivative thereof in a form suitable for conjugating to mRNA encoding another polypeptide.

Other variations and modifications

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein. The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts which are incorporated herein by reference: 1. Sambrook, Fritsch & Maniatis, Molecular Cloning: A Laboratory Manual, Cold

Spring Harbor Laboratories, New York, Third Edition, 2001, whole of Vols I, II, and III;

2. DNA Cloning: A Practical Approach, Vols. I and II (D. N. Glover, ed., 1985), IRL Press, Oxford, whole of text;

3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;

4. Nucleic Acid Hybridization: A Practical Approach (B. D. Hames & S. J. Higgins, eds., 1985) IRL Press, Oxford, whole of text;

5. Animal Cell Culture: Practical Approach, Third Edition (John R.W. Masters, ed., 2000), ISBN 0199637970, whole of text;

6. Immobilized Cells and Enzymes: A Practical Approach (1986) IRL Press, Oxford, whole of text;

7. Perbal, B., A Practical Guide to Molecular Cloning (1984); 8. Methods In Enzymology (S. Colowick and N. Kaplan, eds., Academic Press, Inc.), whole of series;

9. J.F. Ramalho Ortigao, “The Chemistry of Peptide Synthesis” In: Knowledge database of Access to Virtual Laboratory website (Interactiva, Germany);

10. Sakakibara, D., Teichman, J., Lien, E. Land Fenichel, R.L. (1976). Biochem. Biophys. Res. Commun. 73 336-342 11. Merrifield, R.B. (1963). J. Am. Chem. Soc. 85, 2149-2154.

12. Barany, G. and Merrifield, R.B. (1979) in The Peptides (Gross, E. and Meienhofer, J. eds.), vol. 2, pp. 1-284, Academic Press, New York.

13. Wunsch, E., ed. (1974) Synthese von Peptiden in Houben-Weyls Metoden der s Organischen Chemie (Maier, E., ed.), vol. 15, 4th edn., Parts 1 and 2, Thieme, Stuttgart.

14. Bodanszky, M. (1984) Principles of Peptide Synthesis, Springer-Verlag, Heidelberg.

15. Bodanszky, M. & Bodanszky, A. (1984) The Practice of Peptide Synthesis, Springer-Verlag, Heidelberg. 16. Bodanszky, M. (1985) Int. J. Peptide Protein Res. 25, 449-474.

17. Handbook of Experimental Immunology, Vols. I-IV (D. M. Weir and C. C. Blackwell, eds., 1986, Blackwell Scientific Publications).

18. McPherson et al., In: PCR A Practical Approach., IRL Press, Oxford University Press, Oxford, United Kingdom, 1991. 19. Stears et al. (2003) “Trends in microarray analysis” Nature Medicine 9, 140-145.

20. He et al., “Ribosome Display: Cell-free protein display technology” Briefings in Functional genomics and proteomics 1, 204-212, 2002.

The present invention is further described with reference to the following non-limiting examples.

Examples

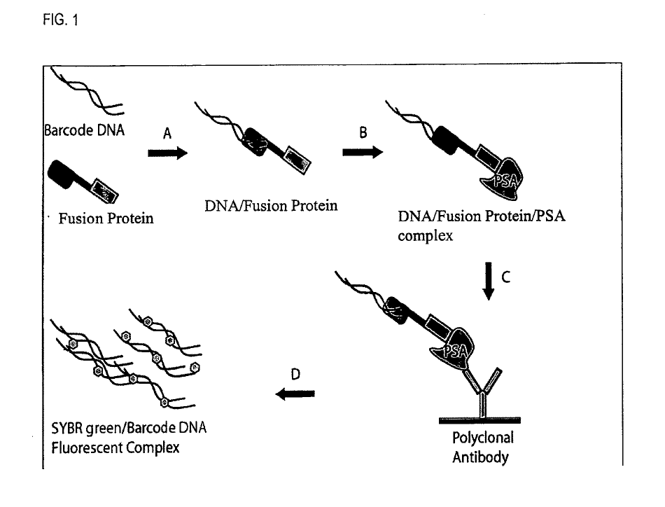

Example 1: Prostate cancer diagnostic system The attributes of the prostate cancer diagnostic system include the linking of a protein (in this case a Ter binding protein fused with an anti-PSA antibody, wherein PSA is prostate specific antigen) to a DNA molecule (in this case Barcode DNA) which is used as part of a signal generation system. The diagnostic system is schematically laid out in FIG. 1 which specifically exemplifies:

(a) a fusion protein or conjugate comprising an anti-target molecule fused to a Ter binding polypeptide which is interacting with Barcode DNA (that is a DNA fragment optimally about 70 by in length containing the TT-lock sequence);

(b) binding of PSA with the complex in (a) and further interacting with a (c) capture surface comprised of polyclonal antibodies raised against PSA covalently bound in a 96-well plate setup; and (d) a signal amplification system of the Barcode DNA using real-time PCR.

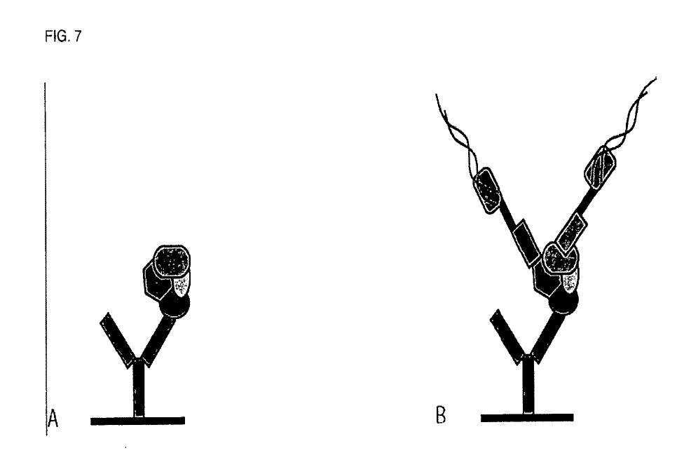

As exemplified herein, the diagnostic system is shown to specifically detect PSA (Bradford et al., Urol Oncol 24:538-551, 2006), a biomarker (target molecule) widely used for screening for prostate cancer. To quantify small amounts of PSA from a complex matrix like human serum, 70 by DNA fragment (Barcode DNA) is used which self assembles with the Ter binding portion of the fusion protein or conjugate with subsequent amplification of this signal using real-time PCR (Klein, Trends Mol Med 8:257-260, 2002) thus PSA can reproducibly be detected and quantified from a complex matrix like human serum as shown in FIG. 1. As shown in FIG. 7, the present invention demonstrates the utility of the diagnostic system to the simultaneous detection and quantification io of different target molecules presented on a surface in a multiplex format.

Example 2: Methods for production of fusion proteins or conjugates

Example 2A: Cloning The Tus, GFP (Tsien, Annu Rev Biochem 67:509-544, 1998) and Tus-GFP fusion genes are expressed under the control of such promoters as bacteriophage T7 or lambda promoters.

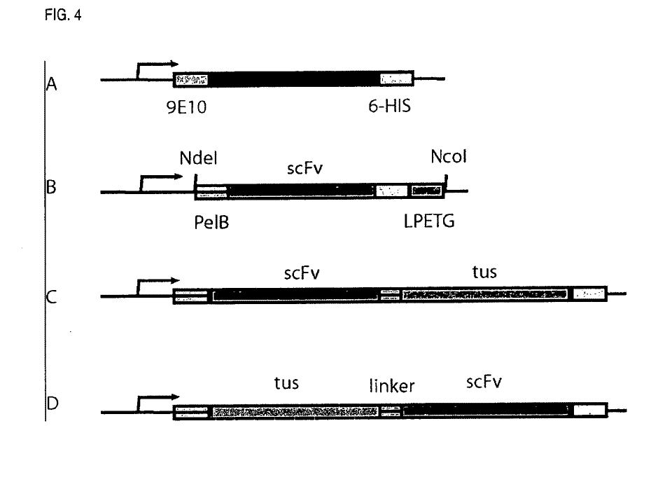

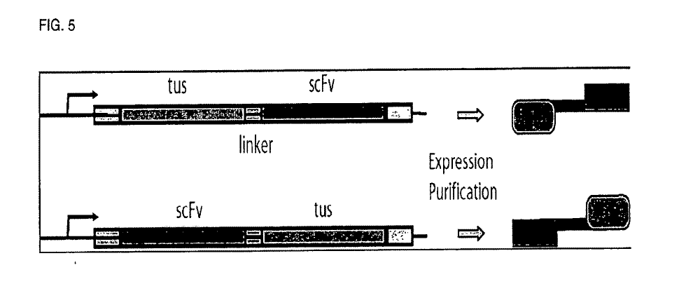

Further to this, the artificial antigen consisting of a human c-myc 9E10 epitope (amino acid sequence EQKLISEEDLN; Schiweck et al., FEBS Lett 414:33-38, 1997; Hilpert et al., Protein Eng 14:803-806, 2001) is N-terminally fused to a C-terminally His6 tagged soluble protein and cloned in a T7-promoter vector pETMCSI (FIG. 4A). The His6 tag is used to immobilize the 9E10 epitope using an anti His6 capture antibody. An E. coli codon optimized version of the gene encoding the anti-c-myc 9E10 scFv with Ndel-Ncol cloning sites, a pelB leader sequence at the N-terminus and a His6 tag at the C-terminus followed by an LPETG tag is cloned alone or as a fusion gene in- frame downstream or upstream of the tus gene (FIG. 4B, C and D). Previous experience shows the vector of choice for the cloning of the fusion genes is pETMCSI (Neylon et al., Biochemistry 39:11989-11999, 2000).

Example 2B: Linkage between the scFv and Tus The present inventors are producing a soluble fusion protein consisting of Tus and the recombinant antibody fragment scFv 9E10 that binds specifically to the c-myc 9E10 epitope (Fuchs et al., Hybridoma 16:227-233, 1997; see FIG. 4A). This can be achieved from expression in the periplasmic space of E. coli of various fusion genes, consisting of the pelB secretion signal (Power et al., Gene 113:95-99, 1992), the scFv 9E10, a flexible linker sequence, and a C- terminally His6-tagged Tus, under the control of the T7 promoter (see FIGS. 4C).

The construct with the scFv and Tus sequence in reverse order (see FIG. 4D) is being expressed. The fusion proteins will then be purified using Ni-NTA affinity chromatography. The position of Tus in the fusion protein may be at the N- or C-terminus and the composition of the flexible linker separating the two domains (e.g., (GGGS)n) may be varied (see FIG. 5).

The N-terminal PelB sequence (Power et al., Gene 113:95-99, 1992) directs the protein into the periplasm (see FIG. 4B). The C-terminal His6 tag is followed by the sortase recognition -

LPETG sequence (see FIG. 4B).

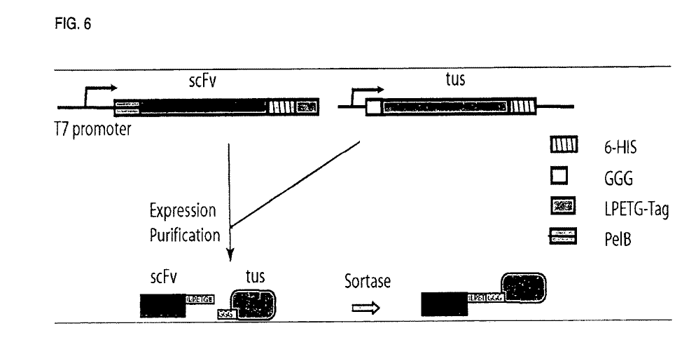

The enzyme sortase is used for efficient ligation of the two proteins. A sequence coding for an N-terminal GGG- tag is fused in frame with the tus gene (FIG. 6) and cloned in pETMCSI. The GGG-Tus is expressed and purified by Ni-NTA affinity chromatography. The ligation of io purified Tus and the scFv 9E10 is carried out analogously to the method described by Mao et al. J

Am Chem Soc 126:2670-2671, (2004). This alternative is very attractive as the scFv and Tus are expressed and purified using standard protocols, and the same Tus sample can be reused for ligation to many different scFvs.

Example 2C: Linkage between GFP and Tus

The present inventors have successfully produced several Tus fusion proteins including Tus-CAT (chloramphenicol acetyl transferase) and a Tus-GFP (green fluorescent protein) used for the successful detection of anti-GFP antibodies in an immunoassay format. The two genes contain a short unstructured spacer. The overproduction and purification of these fusion proteins may be used for the quantification of antiGFP antibodies.

Example 3: Barcode DNA The DNA molecule used in the present invention for signal generation optimally comprises about 75 bp, including a sequence especially designed to be specific to a given Taqman probe flanked by a 21-bp TT-Lock sequence and another specific sequence. The design of pairs of primers and various Taqman probes with different fluorophores produce of a clean and reproducible signal amplification under various temperature conditions enabling a robust and foolproof detection step.

The terminal 21-bp TT-Lock sequence (modified for increased stability of the Tus complex by incorporation of 5-iodo- or 5-bromo-deoxyuridine instead of thymidine at two positions; Mulcair et al., Cell 125, 1309-1319, 2006) followed by an -50bp sequence optimized so that the primers used for signal amplification are absolutely specific. Example 4: Linkage between the fusion protein containing the Ter binding protein and the TT-Lock

To develop a generic method for linking DNA molecules to affinity proteins and to test its use for immunoassay and microarray applications in a monoplex or multiplex format, the inventors assessed cross-linking for method improvement.

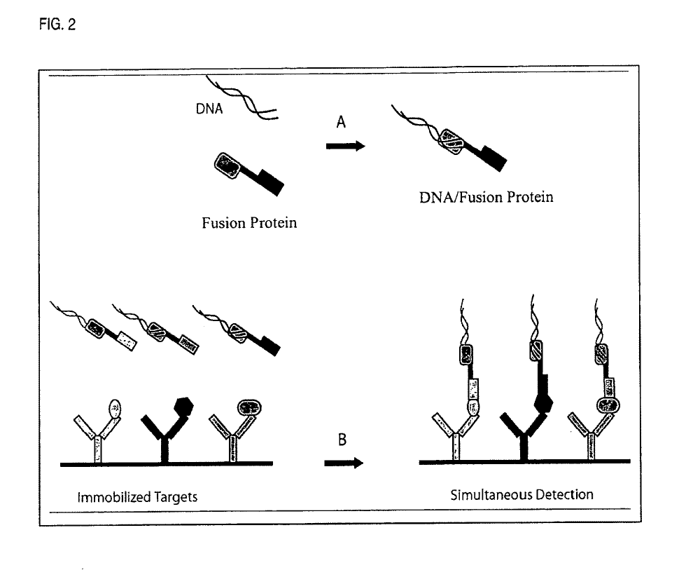

Due to the fast and very stable binding of the TT-Lock to the Tus domain in the fusion protein, it is possible to stoichiometrically bind the DNA containing the 7-Lock sequence to the Tus-anti-target fusion protein for subsequent covalent crosslinking. No purification steps are required at this stage, as the non-bound DNA does not crosslink. The Tus/TT-Lock complex dissociates with a half-life of many hours. Further Tus and TT- Lock variants that will bind irreversibly are engineered. This is guided by current structural knowledge and further structural characterization of a series of Tus/TT-Lock complexes. Different DNAs bind to Tus fusion proteins with different anti-target recognition properties. A mixture of these complexes are able to accurately quantify the different antigens (see FIG. 2) present in a single sample using real-time PCR.

Recent experimental data give the inventors unique capacity in developing a new method for the irreversible and region specific crosslinking of a protein with a DNA molecule that is used for molecular diagnostics in multiplex format.

As described herein, the inventors have surprising discovered an extraordinarily strong interaction between the DNA binding protein Tus and a DNA sequence (7-Lock; Mulcair et al.,

Cell 125, 1309-1319, 2006). The 7-Lock is a partially forked DNA 21-bp sequence that makes an extremely stable interaction with Tus, a monomeric protein from E. coli . This protein-DNA interaction is the strongest of its kind for a monomeric DNA-binding protein with a dissociation half- life of 90 min in 250 mM KCI at 20° C. The DNA sequence can be readily modified further to achieve halflives of at least 10 hours under these high-salt conditions using halogenated nucleotide analogues (Mulcair et al., Cell 125, 1309-1319, 2006).

The inventors as described herein have successfully produced and purified several Tus fusion proteins including a Tus-GFP (green fluorescent protein) that was used for the successful detection of anti-GFP antibodies in an immunoassay format. Further variations of the Ter sequence have been explored herein along with the engineering of irreversible complexes using mild photochemistry for the production of stable complexes and assess the utility of these complexes for the quantification of different target proteins in a multiplex format, as illustrated in FIGS. 2 and 7. Example 5: Crystal structure of Tus and the TT-Lock

Although very stable (KD <0.5 nM), the Tus/TT-Lock complex does dissociate very slowly. This will potentially be a problem for assays in multiplex format. It is therefore desirable to obtain a modified Tus/TT-Lock pair that binds indefinitely under high-salt conditions. Until recently, the most dramatic success in protein-DNA interface engineering has been achieved in phage-display experiments on zinc finger proteins (e.g. Segal et al., Proc Natl Acad Sci USA 96:2758-2763, 2004). Zinc finger proteins are amenable to phage display techniques. DNA recognition is mediated by a small number of residues, well within the library size limits of phage display. The Tus/Ter interactions, however, involve a much larger number of residues that cannot effectively be sampled by phage display libraries. Therefore, a more direct approach to increasing affinity was used.

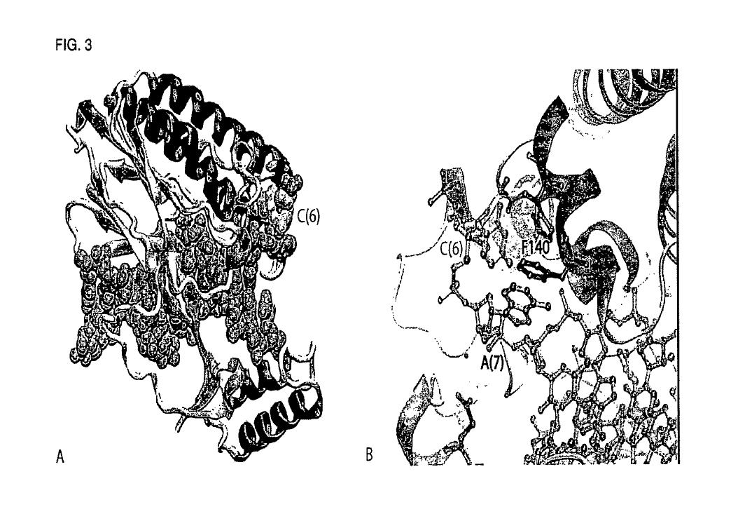

As shown in FIG. 3, the inventors have solved the crystal structure of the Tus/TT-Lock complex, which gives them an unprecedented view of the specific protein-DNA contacts made during this interaction. The crystal structures of the Tus/TerA complex (Kamada et al., Nature 383:598-603, 1996) and the recent crystal structure of the Tus/TT-Lock complex (Mulcair et al., Cell 125, 1309-1319, 2006) aids in the design process.

Example 6: Mutating residues in Tus The present inventors are increasing the binding affinity of Tus to Ter variants by introducing point mutations so as to increase the number of electrostatic interactions at or near the protein/DNA backbone interface. From the crystal structure, the present inventors have identified several neutral or acidic residues close to the phosphate backbone that can be replaced by positively-charged amino acids. Preliminary modelling indicates that these mutated residues should be sterically tolerated in the binding site. Examples are S98K, E125K, T129K, T158K, and N180K. A recently published computational approach to protein/DNA interface design is being used. A module within the ROSETTA program samples a rotomer database of all standard amino acids at an interface and calculates free energies of binding (Havranek et al., J Mol Biol 344:59- 70, 2004). This is used to select further candidate residues for mutagenesis. Each point mutant is being created and tested in a SPR (BIACORE) assay (Mulcair et al., Cell 125, 1309-1319, 2006) before creating a protein containing combinations of mutations with favourable binding properties.

X-ray structure analysis of mutant Tus/Ter and Tus/TT-Lock complexes with high-affinity is being carried out, using conditions for crystallization used in current studies (Mulcair et al., Cell 125, 1309-1319, 2006). This enables the effects of mutations on overall structure to be determined. This information will be used to decide on strategies for further improving affinity of the protein/DNA complexes. Preliminary experiments as described herein have been carried out which demonstrates that an increase in the half-life of the complexes is possible; e.g., the Tus- F140A mutation results in a 10-fold increase in the half-life of the TusfrerB complex as determined by BIACORE experiments (Mulcair et al., Cell 125, 1309-1319, 2006).

Example 7: Use of unnatural nucleotides in the Ter sequence Duggan and co-workers (Biochemistry 35:15391-15396, 1996) showed that substitution of three thymidine bases (T8, T14 and T19) in the Ter sequence with isosteric analogues iodo- and bromo-deoxyuridine (IdU and BrdU) can markedly increase the half-life of the Tus/Ter complex. io These effects have been demonstrated by BIACORE: substitution of T8 and T19 with IdU increases the half-life of the Tus/ter variant complex from 90 to 580 minutes in 250 mM KCI (Mulcair et al., Cell 125, 1309-1319, 2006). Further tests are carried out on the effect of half-life of oligonucleotides with all three of the critical thymidines replaced with IdU. It is thought that this increase is affected by dipole-induced dipole interactions allowed by the electronegative iodine in IdU. Such interactions are not favoured by the methyl group in thymidine. Furthermore, from the

Tus/TT-Lock and Tus/TerA crystal structures, it is anticipated that IdU-containing sequences will allow more van der Waals contacts with Tus compared with thymidine at the equivalent positions. The altered oligonucleotides, which are purchased at little additional cost, are being tested for improved affinity using BIACORE with mutant Ter binding proteins selected above. The present inventors anticipate a synergistic effect between increased electrostatic interactions and the effects of iodouridine substitution.

Example 8: UV crosslinking in multiplex diagnostic format Ultimately, to produce a complex with covalent stability and thus indefinite binding, photochemical crosslinking of a Ter binding protein with a Ter derivative was performed. Previous investigations have demonstrated site-specific crosslinking of BrdU-substituted Ter sites with Tus (Duggan et al., Biochemistry 35:15391-15396, 1996). Efficiency, however, was only 15%.

The Ter binding proteins (Tus, and mutant proteins Tus F140Y and Tus F140W) were expressed and purified using standard methods, as described in Mulcair et al. (2006) Cell, 125: 1309-1319. Stock concentrationswere; Tus (25 microM), Tus F140Y (44 microM) and Tus F140W (5 microM). Tus and Tus F140W were concentrated in a microcon YM10 concentrator to a theoretical concentration of 50 microM.

The following buffers were used in these experiments: Stock buffer: 50 mM Tris (pH 7.6), 1 mM EDTA, 1 mM dithiothreitol, 20% glycerol. UV Buffer: 50 mM Tris (pH 7.6), 250 mM KCI, 0.1 mM EDTA, 0.1 mM dithiothreitol.

The following oligonucleotides (at a stock concentration of 100 p M) were used in these experiments:

RSC838: 5′-GGGGCTATGTTGTAACTAAAG (in 10 mM Tris, 1 mM EDTA, pH 8)

RSC1044: 5′-CTTTAGTTACAACATACTTAT (in 10 mM Tris, 1 mM EDTA, pH 8)

RSC1246: 5′-GGGGAAATGTTGTAACTAAAG (in UV buffer) RSC1249: 5′-CTTTAGTTACAACATXCTTAT (X is 5-Bromodeoxyuridine, BrdU, in UV buffer)

RSC838/1044: 20 microL of oligonucleotide RSC838 and 20 microL of oligonucleotide io RSC1044 were mixed to yield a final concentration of 50 microM each. The mixture was heated up 1 minute at 72 C in an aluminium block and allowed to cool to RT over a period of 5 minutes then stored on ice.

RSC1246/1249: 20 microL RSC1246 and 20 microL RSC1249 were mixed to yield a final concentration of 50 microM each. The mixture was heated for 1 minute at 72 ° C. in an aluminium block and allowed to cool to room temperature over a period of 5 minutes, then stored on ice.

A droplet comprising 3 p L of Ter-binding protein and 3 p L of annealed oligonucleotides is deposited in a 12 well multidish (Nunclon) and left at room temperature for 10 minutes. The 12 well multidish is turned upside down without lid over a transilluminator and irradiated at 312 nm during 5 minutes. A pre-chilled aluminium block (-20 C) is positioned over the dish to avoid overheating. The yield of crosslinking was assessed by SDS-PAGE electrophoresis using a 12.5% nextgel (Amresco).

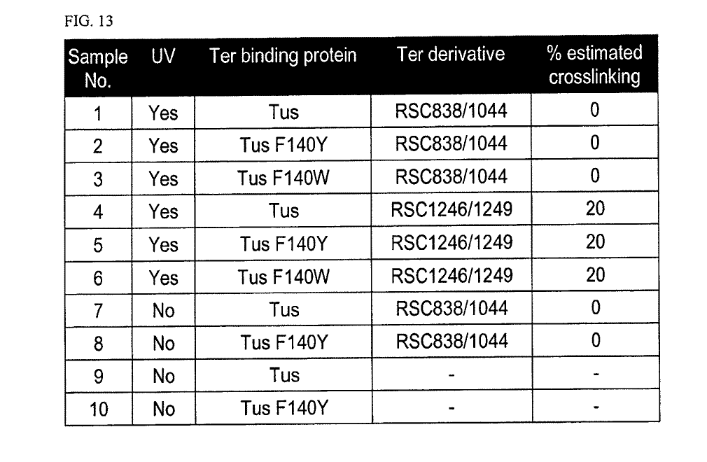

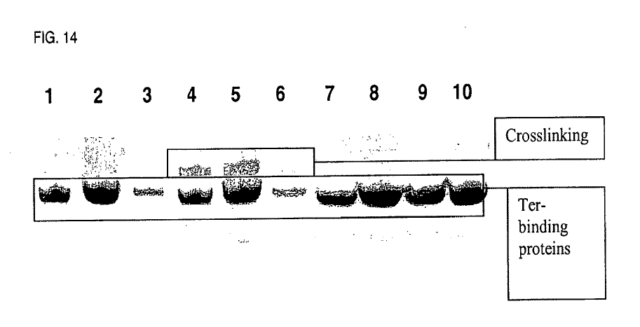

The results as shown in FIGS. 13 and 14 that under the tested conditions only oligonucleotides having a BrdU substitution were able to crosslink with any of the Ter-binding proteins. The crosslinking efficiency was at least about 20%. This efficiency can be further improved using other substituents and conditions. In another experiment the oligonucleotide pair

RSC838/1249 yielded also at least about 20% crosslinked products with Ter-binding proteins under similar conditions. These data demonstrate the utility of this system in multiplex format.

Example 9: Evaluation of diagnostic applications Example 9A: Preliminary tests

The kinetic and thermodynamic parameters of the DNATTus-anti-Target complexes under various temperature and ionic strength conditions using a BIACORE SPR biosensor to define the optimal conditions for this ultrasensitive diagnostic method are being studied. The present inventors are using the BIACORE assay that has been successfully used to study the interaction of Ter with Tus (Neylon et al., Biochemistry 39:11989-11999, 2000; Mulcair et al., Cell 125, 1309- 1319, 2006) to characterize the DNA/Tus-anti-target molecule complexes. The present inventors are also using a slightly different BIACORE-based strategy to characterize the binding of a target protein to the DNA/Tus-anti-Target complexes. The present inventors are first immobilising the Tus-anti-target to a DNA displayed on a streptavidin chip (Biacore). In addition, the formation of the ternary complex upon binding of the target under various conditions to find those best for the method is being characterized as well as the stability and unfolding of Tus-anti-Target and the DNA/Tus-anti-Target complexes. These data will define the limits and optimal storage conditions of the diagnostic components.

Example 9B: Diagnositc test for the detection of an Anti-GFP antibody An assay was developed to detect the presence of a target molecule (biotinylated goat polyclonal antibody (Ab) to GFP) in a sample by using a fusion protein comprising Tus and an anti- target molecule (GFP) linked to a Barcode DNA. The assay also included an immobilized molecule. In this example, streptavidin (streptavidin-coated PCR Tubes) was used as the immobilized molecule to bind to biotin of the target molecule and thus immobilizing the target molecule.

All reagents (target, fusion protein and Barcode DNA) are added together in a one pot reaction for the binding step. After an incubation step and several wash steps the Barcode DNA is detected and quantified by real-time PCR correlating with the presence of target in the sample.

The following is a list of oligonucleotides that were used in this assay:

PSJCU1:
5′-CAGTATGGTGCTTCACACG
PSJCU2:
5′CAGTATGGTGCTTCACACGGATAGATGTTACTTCGCTCTTTAGTTAC
AACATACTTAT
PSJCU3:
5′-TATGTTGTAACTAAAGAGCG

Oligonucleotides were dissolved in water to a final concentration of 100 microM. Tus-GFP (anti-target) protein was expressed from E. coli BL21/pLysS/pPMS1259 that encodes the Tus-GFP fusion gene. The fusion protein was purified through Ni-NTA-agarose chromatography. Green fluorescent fractions were combined and the concentration of the stock was estimated to be 4 microM of anti-target.

Biotinylated goat polyclonal Ab to GFP (target protein: 1mg/ml, Abcam, ab6658), Platinum® SYBR® Green qPCR SuperMix-UDG (Invitrogen) and Streptavidin-coated PCR Tubes (Strips, Roche). Also used in this assay is the “Bind and wash” (BW) buffer which consists of 20 mM Tris (pH 8.0), 150 mM NaCI, 0.005% (v/v) Tween 20.

The Barcode DNA containing the 7-Lock was prepared by diluting oligonucleotides PSJCU2 and PSJCU3 in 20 mM Tris (pH 8.0), 150 mM NaCI to yield a final concentration of 1 microM and 5 p M respectively. The mixture was heated up (80 ° C.) in an aluminium block and allowed to cool to room temperature over a period of 30 minutes to yield a 1 p M solution of Barcode DNA. In preparing the Anti-Target/Barcode DNA complex, 1 p L of anti-target and 5 p L of

Barcode DNA were mixed with 994 microL of BW (final concentrations: 4 nM anti-target and 5 nM Barcode DNA and left at room temperature for 10 minutes.

For the preparation of the target molecule, two dilutions of target were prepared, 50 pg/p L and 0.5 pg/p L, in BW. Every reaction containing different target quantities were set up in the following way:

2 p L of target (100, 1 or 0 pg) were mixed with 18 p L of Anti-Target/Barcode DNA complex in streptavidin-coated PCR Tubes. The mixture was allowed to bind at room temperature for 45 minutes.

After binding the supernatant was discarded followed by a quick centrifugation step to is remove any solution left on the walls of the tubes. This was followed by one wash step with 100 p L of BW. The supernatant was removed followed by a quick centrifugation step to remove any solution left on the walls of the tubes. This was then followed by 3 supplementary wash steps with 130 p L and a last wash step with 200 p L, followed each time by a quick centrifugation step to remove any solution left on the walls of the tubes. At this stage 25 p L of a primer mix solution containing 0.5 p M each of PSJCU1 and

PSJCU3 were added and the samples were heated to 95 ° C. during 3 minutes. These samples were than stored on ice until the quantification step by real-time PCR.

For quantification using real-time PCR, 12.5 p L of sample (half reaction sample) was mixed with 12.5 p L Platinum® SYBR® Green qPCR SuperMix-UDG. The real-time cycler was a Corbett Research Rotor-Gene 3000 (Corbett). The following two tables provide the experimental parameters for real-time PCR.

| Run Name | SYBR Green(R) I 2007-05-29 (1) 2007-05-31 |
|---|---|
| | (2) 2007-06-01 (1) |
| Run On Software | Rotor-Gene 6.1.81 |
| Version | |
| Gain FAM/Sybr | 10. |

| | |
|---|---|
| Threshold | 0.03335 |
| Left Threshold | 0.000 |
| Standard Curve Imported | No |
| Standard Curve (1) | conc = 10\hat{ }(−0.255 * CT + 3.955) |
| Standard Curve (2) | CT = −3.917 * log(conc) + 15.491 |
| Reaction efficiency (*) | 0.80021 (* = 10\hat{ }(−1/m) − 1) |
| M | −3.91661 |
| B | 15.4912 |
| R Value | 0.99997 |
| R\hat{ }2 Value | 0.99994 |
| Start normalising from cycle | 1 |
| Noise Slope Correction | No |
| No Template Control Threshold | 0% |
| Reaction Efficiency Threshold | Disabled |
| Normalisation Method | Dynamic Tube Normalisation |
| Digital Filter | Light |

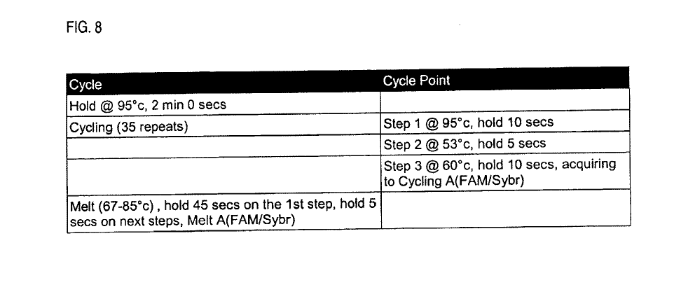

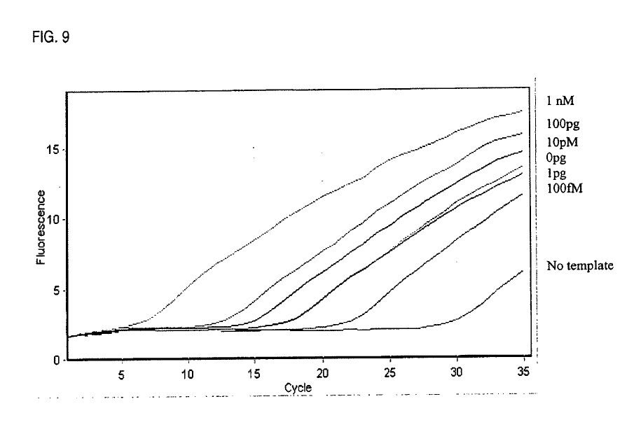

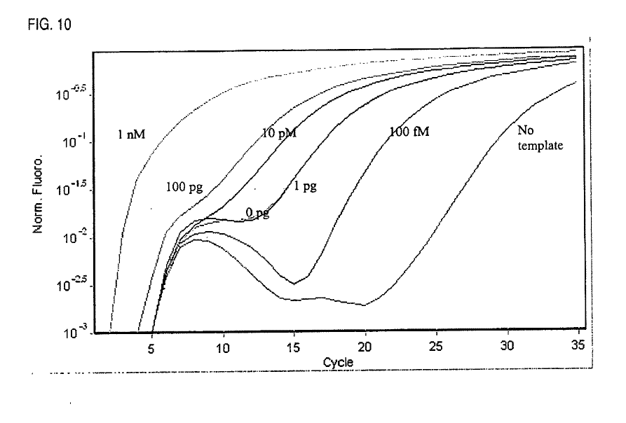

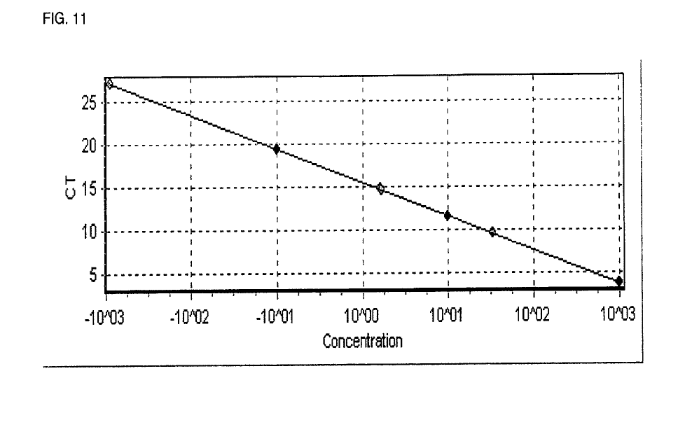

In addition, the cycling parameters of the PCR are provided in FIG. 8. The fluorescent intensities of the samples subject to real-time PCR are shown in FIGS. 9 and 10 which represent raw and normalized log-transformed raw data respectively generated by the Rotor-Gene 6.1.81 software package. A standard curve was generated from the normalized log-transformed raw data by the Rotor-Gene 6.1.81 software package as shown in FIG. 11.

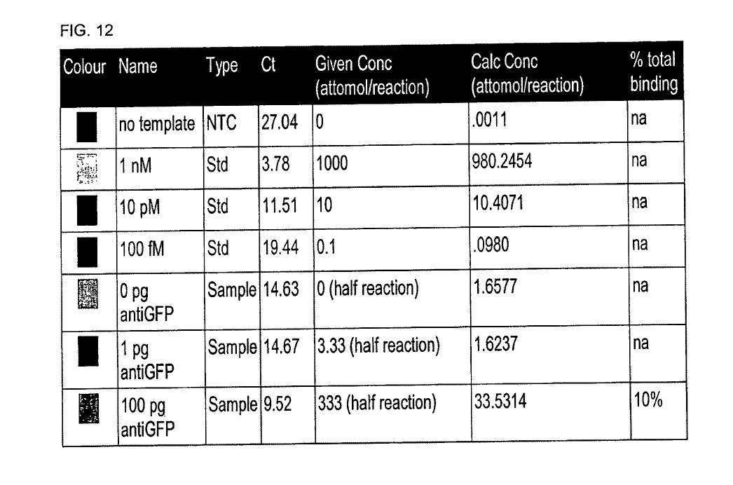

I0 The results as represented in FIG. 12 show that under the tested conditions described herein, the background level of the diagnostic corresponds about 5 pg per sample of target and that 100 pg of target (biotinylated goat polyclonal Ab to GFP) can easily be quantified using this assay. The assay is performed in less than 2 hours and the limit of detection can be improved using more dilute conditions, more sample volume and/or additives for the elimination of non- specific binding of Barcode DNA.

Example 9C: Evaluation in multiplex format Ultimately the present application shows that multiple antigens or pathogens can be quantified at the same time from a single sample. This is a great advantage because it would drastically reduce the costs and time for accurate diagnosis. The present inventors are immobilising a mixture of commercially available monoclonal anti-GFP antibodies and c-myc tagged protein as the immobilised target molecules in a 96-well plate. This is achieved using a mixture of specific capture antibodies. The present inventors then use a mixture of DNATTus-GFP and DNATTus-scFv 9E10 for detection and multiplex real-time PCR for quantification. A mixture of these complexes is tested to determine if they accurately quantify different antigens present in a sample without cross-reactivity (see FIG. 2).

As shown in FIGS. 2 and 7, the method and process of detecting and/or quantifying target molecules in a multiplex format can also be used as a convenient alternative to techniques such as immunoprecipation followed by Western blotting.

1. A method of detecting and/or quantifying a target molecule from a sample obtained from a subject wherein the method comprises:
(i) incubating a fusion protein or conjugate comprising a Ter binding polypeptide fused to at least one anti-target molecule or fragment thereof with a partially double-stranded oligonucleotide comprising a barcode DNA sequence for a time and under conditions sufficient to bind to said Ter binding polypeptide thereby producing a complex;
(ii) incubating said complex in the presence of said sample comprising said target molecule for a time and under conditions sufficient for said anti- target molecule to bind to said target molecule thereby producing a target- bound complex;
(iii) incubating said target-bound complex in the presence of at least one immobilised molecule wherein said immobilised molecule has an affinity to said target molecule;
(iv) incubating said immobilised molecule for a time and under conditions sufficient to bind to said target molecule thus immobilising said target molecule; and
(v) detecting and/or quantifying said target molecule.

2-11. (canceled)

12. The method according to any-ene-of claims 1 to-11 wherein the double-stranded oligonucleotide comprises a first strand and a second strand, wherein:
(a) said first strand comprises the sequence:
5′-Nc R N_{D} G T T G T A AC N_{D} A-3′ (SEQ ID NO: 1) or an analogue or derivative of said sequence; and
(b) said second strand comprises the sequence:
5!-T N_{D} GT TACA AC N_{D} T Nc C-3′ (SEQ ID NO: 2) or an analogue or derivative of said sequence wherein R is a purine, N_{c} and N_{D} are each a DNA or RNA residue or analogue thereof, N_{D} residues in said first strand and said second strand are sufficiently complementary to permit said N_{D} residues to be annealed in the double-stranded oligonucleotide, and the sequence 5′- GTTGTAAC-3′ (SEQ ID NO: 3) of said first strand is annealed to the complementary sequence 5′-GTTACAAC-3′ (SEQ ID NO: 4) of said second strand.

13. The method according to an-ene-ef claims 1 tem wherein the double-stranded oligonucleotide comprises a first strand and a second strand wherein:
(a) said first strand comprises the sequence:
⁵¹⁻(N_{A})ᵢₙ N_{E} N_{E} N_{B} N_{B} Nc R N_{D} GT TGT AA CN_{D} A (N_{A}).-3′ (SEQ ID NO: 55), N_{c —}1M GTT GT A AC (SEQ ID NO: 57) NE N_{E—}NaNja NcRTGTTGTAACTAAAG-3′(SEQIDNO:
581 or an analogue or derivative of said sequence; and
(b) said second strand comprises the sequence:
5′-(1\T_{A})ₚ T N_{D} GTTACAAC N_{D} T Nc C N_{B} N_{E} N_{E} (N_{A})₀^{-3!} (SEQ ID NO: 56) 5′-_{—A}j₃TAGTTACAACATACN_{B} NE (SEQIDNO: 59)or 5′-CTTTAGTTACAACATACN_{R} N_{E} N_{F} (N_{A})₁-₁₅⁻3^{′} (SEQIDNO: 60)or an analogue or derivative of said sequence wherein N_{A}, N_{B} and N_{E} are each any DNA or RNA residue or analogue thereof, each of N_{A} and N_{B} is optional subject to the proviso that when any occurrence of N_{B} is present it is not base- paired to another residue, base-pairing of each of N_{c} to another residue is optional, each of N_{D} is base-paired with another residue, each of N_{E} is optional, subject to the proviso that if one or more of N_{E} is present it is not base-paired unless m=0 or o=0, m, n, o, p, are each an integer including zero, and said first strand and said second strand are of equal or unequal length.

14. The method according to any one of claims 1 to-13 wherein the oligonucleotide is forked.

15. (canceled)

16. The method according to claim 12 15 wherein the analogue comprises a methylated, iodinated, brominated or biotinylated residue.

17-22. (canceled)

23. The method according to any one of claims 1 to-22 wherein the oligonucleotide is contained in a Barcode DNA sequence.

24. The method according to any-ene-ef claims 1 to 23 wherein the oligonucleotide binds to a Ter binding polypeptide covalently or non-covalently.

25. The method according to claim 24 wherein the Ter binding polypeptide has TerB-binding activity.

26. The method according to claim 25 wherein the Ter binding polypeptide comprises the sequence set forth as SEQ ID NO: 5.

27-31. (canceled)

32. The method according to any one of claims 1 te--3-1 wherein the method comprises a chip comprising said oligonucleotide.

33. The method according to any one of claims 1 tem wherein the target molecule is a biological marker (biomarker) for the detection or indication of a disease or condition.

34-42. (canceled)

43. The method according to an^{,}_{f}rene-ef-claims 1 to-42 wherein the anti-target molecule comprises an antigen, antibody, or any other molecule that has an affinity to the target molecule.

44. The method according to any one of claims 1 to-43 wherein the target molecule is detected and/or quantified by use of a signal molecule bound to a Ter binding polypeptide or derivative, analogue or fragment thereof wherein the fragment possesses Ter binding activity, Ter or TTLock or derivatives or analogues thereof, and/or said anti-target molecules.

45. The method according to claim 44 wherein the signal molecule comprises a coloured compound, a fluorescent tag, an intercalating dye or a radioactive isotope or a combination thereof.

46-85. (canceled)

86. A kit for detecting a target molecule from a sample of a subject in a monoplex or multiplex format comprising a first strand oligonucleotide or an analogue or derivative thereof, and a second strand oligonucleotide or an analogue or derivative thereof, wherein said first strand oligonucleotide or analogue or derivative and said second strand oligonucleotide or analogue or derivative are in a form suitable for their annealing to produce a partially double-stranded oligonucleotide wherein:
(a) said first strand comprises the sequence:
R N_{D} OTT GT A AC N_{D} A-3′ (SEQ ID NO: 1) or an analogue or derivative of said sequence; and
(b) said second strand comprises the sequence:
5′-T N_{D} GT T AC A AC N_{D} T Nc C-3′ (SEQ ID NO: 2) or an analogue or derivative of said sequence
wherein R is a purine, N_{c} and N_{D} are each a DNA or RNA residue or analogue thereof, N_{D} residues in said first strand and said second strand are sufficiently complementary to permit said N_{D} residues to be annealed in the double-stranded oligonucleotide, and the sequence 5′- GTTGTAAC-3′ (SEQ ID NO: 3) of said first strand is annealed to the complementary sequence 5′-GTTACAAC-3′ (SEQ ID NO: 4) of said second strand in a form suitable for conjugating to a second molecule, wherein said second molecule comprises a nucleic acid, polypeptide or small molecule.

87. (canceled)

88. The kit according to claim 87 86 wherein the second molecule is a Ter binding polypeptide.

89. The kit according to any one of claims 86 to-8-8- wherein:
(a) said first strand comprises the sequence:
5^{′}4N_{A})ₘ N_{E} N_{E} N_{B} N_{B} Nc R N_{D} OTTGTAAC N_{D} A (N_{A})ₙ-3′ (SEQ ID NO: 55) 5′- WA)1-15 N_E NF NB NB Nc R N_{D} GTTGTAAC N_{T}A_{A})3-3′ (SEQ ID NO: 57), Nc RTGTTGTAACTAAA G-3′ (SEQ ID NO: 58) or an analogue or derivative of said sequence; and
(b) said second strand comprises the sequence:
5′-(N_{A})ₚ T N_{D} GT T A C A A C N_{D} T Nc C N_{B} N_{E} N_{E} (N_{A})ₒ-3′ (SEQ ID NO: 56) 5′- ffj,j3 TAGTTACAACATAC N_{B} (SEQ ID NO: 59) or 5′-C TTTAGTTACAACATACN_{R} N_{F}N_{F—}ff_{—.},₁)₁₋₁₅-3′(SEQIDNO: 60)oran analogue or derivative of said sequence
wherein N_{A}, N_{B} and N_{E} are each any DNA or RNA residue or analogue thereof, each of N_{A} and N_{B} is optional subject to the proviso that when any occurrence of N_{B} is present it is not base- paired to another residue, base-pairing of each of N_{c} to another residue is optional, each of N_{D} is base-paired with another residue, each of N_{E} is optional, subject to the proviso that if one or more of N_{E} is present it is not base-paired unless m=0 or o=0, m, n, o, p, are each an integer including zero, and said first strand and said second strand are of equal or unequal length.

90. The kit according to any one of claims 86 to 89 wherein the oligonucleotide is forked.

91-98. (canceled)

99. The kit according to any one of claims 86 te-98- wherein the oligonucleotide is contained in a Barcode DNA sequence.

100. The method according to claim 13 wherein the analogue comprises a methylated, iodinated, brominated or biotinylated residue.
